SEQUENCE LISTING (60 sequences)

SEQ ID NO: 1 - protein, 484 aa, Artificial Sequence
  Recombinant human trpRS
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQKLAAALEHHHHHH

SEQ ID NO: 2 - DNA, 4877 bp, Artificial Sequence
  Recombinant human mini-TrpRS nucleotide construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagaccacaacggtttccctctagaaataattttgtttaactttaagaaggagatatacatatgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcagaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 3 - protein, 437 aa, Artificial Sequence
  Recombinant human mini-TrpRS protein construct
MSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQKLAAALEHHHHHH

SEQ ID NO: 4 - DNA, 4811 bp, Artificial Sequence
  Recombinant human Met-T1-His tag nucleotide construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagaccacaacggtttccctctagaaataattttgtttaactttaagaaggagatatacatatgagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcagaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 5 - protein, 415 aa, Artificial Sequence
  Recombinant human Met-T1-His tag protein construct
MSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQKLAAALEHHHHHH

SEQ ID NO: 6 - DNA, 4742 bp, Artificial Sequence
  Recombinant human Met-T2-His tag nucleotide construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagaccacaacggtttccctctagaaataattttgtttaactttaagaaggagatatacatatgagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcagaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 7 - protein, 392 aa, Artificial Sequence
  Recombinant human Met-T2-His tag protein construct
MSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQKLAAALEHHHHHH

SEQ ID NO: 8 - protein, 5 aa, Homo sapiens
SNHGP

SEQ ID NO: 9 - protein, 5 aa, Homo sapiens
SAKGI

SEQ ID NO: 10 - protein, 4 aa, Homo sapiens
HVGH

SEQ ID NO: 11 - protein, 5 aa, Homo sapiens
KMSAS

SEQ ID NO: 12 - protein, 378 aa, Homo sapiens
SAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 13 - protein, 401 aa, Homo sapiens
SNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 14 - protein, 423 aa, Artificial Sequence
  Recombinant human mini-TrpRS (YG variant) construct
SYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 15 - protein, 379 aa, Artificial Sequence
  Recombinant human Met-T2 (YG variant) construct
MSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 16 - protein, 402 aa, Artificial Sequence
  Recombinant human Met-T1 (YG variant) construct
MSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 17 - protein, 424 aa, Artificial Sequence
  Recombinant human Met-mini-TrpRS (YG variant) construct
MSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 18 - DNA, 1134 bp, Artificial Sequence
  Recombinant human T2 (YG variant) construct
agtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 19 - DNA, 1137 bp, Artificial Sequence
  Recombinant human Met-T2 (YG variant) construct
atgagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 20 - DNA, 1203 bp, Artificial Sequence
  Recombinant human T1 (YG variant) construct
agtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 21 - DNA, 1206 bp, Artificial Sequence
  Recombinant human Met-T1 (YG variant) construct
atgagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 22 - DNA, 1269 bp, Artificial Sequence
  Recombinant human mini-TrpRS (YG variant) construct
agctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 23 - DNA, 1272 bp, Artificial Sequence
  Recombinant human Met-mini-TrpRS (YG variant) construct
atgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 24 - protein, 378 aa, Artificial Sequence
  Recombinant human T2 (SG variant) construct
SAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 25 - protein, 401 aa, Artificial Sequence
  Recombinant human T1 (SG variant) construct
SNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 26 - protein, 423 aa, Artificial Sequence
  Recombinant human mini-TrpRS (SG variant) construct
SYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 27 - protein, 379 aa, Artificial Sequence
  Recombinant human Met-T2 (SG variant) construct
MSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 28 - protein, 402 aa, Artificial Sequence
  Recombinant human Met-T1 (SG variant) construct
MSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 29 - protein, 424 aa, Artificial Sequence
  Recombinant human Met-mini-TrpRS (SG variant) construct
MSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 30 - DNA, 1134 bp, Artificial Sequence
  Recombinant human T2 (SG variant) construct
agtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnnggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 31 - DNA, 1137 bp, Artificial Sequence
  Recombinant human Met-T2 (SG variant) construct
atgagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnnggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 32 - DNA, 1203 bp, Artificial Sequence
  Recombinant human T1 (SG variant) construct
agtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnnggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 33 - DNA, 1206 bp, Artificial Sequence
  Recombinant human Met-T1 (SG variant) construct
atgagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnnggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 34 - DNA, 1269 bp, Artificial Sequence
  Recombinant human mini-TrpRS (SG variant) construct
agctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnnggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 35 - DNA, 1272 bp, Artificial Sequence
  Recombinant human Met-mini-TrpRS (SG variant) construct
atgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnnggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 36 - protein, 378 aa, Artificial Sequence
  Recombinant human T2 (YD variant) construct
SAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 37 - protein, 401 aa, Artificial Sequence
  Recombinant human T1 (YD variant) construct
SNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 38 - protein, 423 aa, Artificial Sequence
  Recombinant human mini-TrpRS (YD variant) construct
SYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 39 - protein, 379 aa, Artificial Sequence
  Recombinant human Met-T2 (YD variant) construct
MSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 40 - protein, 402 aa, Artificial Sequence
  Recombinant human Met-T1 (YD variant) construct
MSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 41 - protein, 424 aa, Artificial Sequence
  Recombinant human Met-mini-TrpRS (YD variant) construct
MSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 42 - DNA, 1134 bp, Artificial Sequence
  Recombinant human T2 (YD variant) construct
agtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctaygaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 43 - DNA, 1137 bp, Artificial Sequence
  Recombinant human Met-T2 (YD variant) construct
atgagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctaygaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 44 - DNA, 1203 bp, Artificial Sequence
  Recombinant human T1 (YD variant) construct
agtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctaygaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 45 - DNA, 1206 bp, Artificial Sequence
  Recombinant human Met-T1 (YD variant) construct
atgagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctaygaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 46 - DNA, 1269 bp, Artificial Sequence
  Recombinant human mini-TrpRS (YD variant) construct
agctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctaygaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 47 - DNA, 1272 bp, Artificial Sequence
  Recombinant human Met-mini-TrpRS (YD variant) construct
atgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctaygaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 48 - protein, 378 aa, Artificial Sequence
  Recombinant human T2 (SD variant) construct
SAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 49 - protein, 401 aa, Artificial Sequence
  Recombinant human T1 (SD variant) construct
SNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 50 - protein, 423 aa, Artificial Sequence
  Recombinant human mini-TrpRS (SD variant) construct
SYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 51 - protein, 379 aa, Artificial Sequence
  Recombinant human Met-T2 (SD variant) construct
MSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 52 - protein, 402 aa, Artificial Sequence
  Recombinant human Met-T1 (SD variant) construct
MSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 53 - protein, 424 aa, Artificial Sequence
  Recombinant human Met-mini-TrpRS (SD variant) construct
MSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQASDDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 54 - DNA, 1134 bp, Artificial Sequence
  Recombinant human T2 (SD variant) construct
agtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnngaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 55 - DNA, 1137 bp, Artificial Sequence
  Recombinant human Met-T2 (SD variant) construct
atgagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnngaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 56 - DNA, 1203 bp, Artificial Sequence
  Recombinant human T1 (SD variant) construct
agtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnngaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 57 - DNA, 1206 bp, Artificial Sequence
  Recombinant human Met-T1 (SD variant) construct
atgagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnngaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 58 - DNA, 1269 bp, Artificial Sequence
  Recombinant human mini-TrpRS (SD variant) construct
agctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnngaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 59 - DNA, 1272 bp, Artificial Sequence
  Recombinant human Met-mini-TrpRS (SD variant) construct
atgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggccnnngaygatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcag

SEQ ID NO: 60 - protein, 56 aa, Homo sapiens
DLDYMGMSSGFYKNVVKIQKHVTFNQVKGIFGFTDSDCIGKISFPAIQAAPSFSNS

(19) United States
(12) Patent Application Publication
(10) Pub. No.: US 2006/0024286 A1
(43) Pub. Date: Feb. 2, 2006

(54) Variants of tRNA synthetase fragments and uses thereof

(76) Inventor: Paul Glidden, San Diego, CA (US)

Correspondence Address: ELSA DJUARDI;PFIZER, INC., 10777 SCIENCE CENTER DRIVE, SAN DIEGO, CA, 92121, US

(21) Appl. No.: 10/980,866

(22) Filed: Nov. 2, 2004

Related U.S. Application Data
(63) Continuation-in-part of application No. 10/962,218, filed on Oct. 7, 2004.
(60) Provisional application No. 60/598,019, filed on Aug. 2, 2004.

(51) Int. Cl. A61K 38/47; C12N 9/22

(52) U.S. Cl. 424/94.61; 435/199

(57) ABSTRACT

The present invention relates to compositions and methods for treating conditions associated with angiogenesis. In particular the present invention relates to variants of tRNA synthetase fragments, or more preferably tryptophanyl tRNA synthetase fragments, or more preferably human tryptophanyl tRNA synthetase fragments.

### CROSS REFERENCES

This application claims priority to U.S. Provisional Application No. 60/598,019 filed on Aug. 2, 2004 and is a continulation-in-part of U.S. application Ser. No. 10/962,218, which was filed on Oct. 7, 2004.

### BACKGROUND

Normal tissue growth, which occurs during embryonic development, wound healing, and menstrual cycle is characterized by dependence on new vessel formation for the supply of oxygen and nutrients as well as removal of waste products. Angiogenesis is the name given to the development of new capillaries from pre-existing blood vessels. The extent of angiogenesis is determined by the balance between pro-angiogenic factors and anti-angiogenic factors. Pro-angiogenic factors include, but are not limited to, vascular endothelial growth factor (VEGF), fibroblast growth factor (FGF), interleukin-8 (IL-8), angiogenin, angiotropin, epidermal growth factor (EGF), platelet derived endothelial cell growth factor, transforming growth factor α (TGF-α), transforming growth factor β (TGF-β), and nitric oxide. Anti-angiogenic factors include, but are not limited to, thrombospondin, angiostatin, and endostatin.

While in most normal tissues the balance favors the anti-angiogenic factors and angiogenesis is inhibited, numerous conditions may become manifested upon a switch to an angiogenesis-stimulating phenotype. Such angiogenic conditions include, but are not limited to, age-related macular degeneration (AMD), cancer (both solid and hematologic), developmental abnormalities (organogenesis), diabetic blindness, endometriosis, ocular neovascularization, psoriasis, rheumatoid arthritis (RA), skin disclolorations (e.g., hemangioma, nevus flammeus, or nevus simplex) and wound healing.

It is desirable to identify compositions and methods that modulate or inhibit angiogenesis.

### SUMMARY OF THE INVENTION

The present invention relates to a composition comprising an isolated tRNA synthetase fragment, wherein the tRNA synthetase fragment comprises, consists essentially of, or consists of an amino acid sequence SEQ ID NO: 12, 15, 24, 27, 36, 39, 48 or 51. Preferably, such tRNA synthetase fragment is less than 45 kD, more preferably less than 44 kD, less than 43.9 kD, 43.8 kD, 43.7 kD, 43.6 kD, or more preferably less than 43.5 kD. Preferably such tRNA synthetase fragment is anti-angiogenic.

In some embodiments, the present invention relates to a composition comprising an isolated tRNA synthetase fragment, wherein the tRNA synthetase fragment comprises, consists essentially of, or consists of SEQ ID NO: 13, 16, 25, 28, 37, 40, 49 or 52. Preferably, such tRNA synthetase fragment is less than 48 kD, more preferably less than 47 kD, or more preferably less than 46 kD. Preferably such tRNA synthetase fragment is anti-angiogenic.

In some embodiments, the present invention relates to a composition comprising an isolated tRNA synthetase fragment, wherein the tRNA synthetase fragment comprises, consists essentially of, or consists of SEQ ID NO: 14, 17, 26, 29, 38, 41, 50 or 53. Preferably, such tRNA synthetase fragment is less than 53 kD, more preferably less than 52 kD, more preferably less than 51 kD, more preferably less than 50 kD, or more preferably less than 49 kD. Preferably such tRNA synthetase fragment is anti-angiogenic.

In any of the embodiments herein, a tRNA synthetase fragment is preferably isolated. In any of the embodiments herein, a tRNA synthetase fragment is preferably purified. Such purification step may reduce the amout of an endotoxin in a pharmaceutical composition. In some embodiments, the amout of endotoxin in a composition is less than 30, 20, 10, or more preferably 9, 8, 7, 6, 5, 4, 3, 2, or 1 endounits.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the amino acid residue sequence of tryptophanyl-tRNA synthetase polypeptide (SEQ ID NO: 1) with signature sequences (SEQ ID NO: 10 & SEQ ID NO: 11), shown in a box, which is also encompassed within the truncated form of tryptophanyl tRNA synthetase (amino acid residue sequences 94-471 of SEQ ID NO: 1).

FIG. 2 is a photomicrograph that illustrates retinal vascular development in a mouse model.

FIG. 3 is a graphical representation of data reported in Example 3, below.

FIG. 4 is a graphical representation of data reported in Example 4, below.

FIG. 5 is a photomicrograph that illustrates the binding localization of his-tagged T2 (SEQ ID NO: 7) in the retina in a mouse model.

FIG. 6 illustrates pI of a polypeptide recombinantly produced by an expression vector encoding a polypeptide of SEQ ID NO: 15.

FIG. 7 illustrates a flowchart illustration of one possible method for purifying the compositions herein.

FIG. 8 illustrates another embodiment of the purification methods of the invention.

FIG. 9 illustrates an SDS-Page analysis demonstrating purity of a polypeptide produced by a bacteria host cell transfected with a polynucleotide encoding SEQ ID NO: 15 and further purified using one of the methods herein.

### DETAILED DESCRIPTION OF THE INVENTION

I. Definitions

The term “amino acid” or “amino acid residue” refers to an amino acid which is preferably in the L-isomeric form. When an amino acid residue is part of a polypeptide chain, the D-isomeric form of the amino acid can be substituted for the L-amino acid residue, as long as the desired functional property is retained. NH₂ refers to the free amino group present at the amino terminus of a polypeptide. COOH refers to the free carboxy group present at the carboxyl terminus of a polypeptide.

In keeping with standard polypeptide nomenclature described in J. Biol. Chem., 243:3552-59 (1969) and adopted at 37 C.F.R. §§ 1.821-1.822, all amino acid residue sequences represented herein by formulae have a left to right orientation in the conventional direction of amino-terminus to carboxyl-terminus. In addition, the phrase “amino acid residue” is broadly defined to include modified and unusual amino acids, such as those referred to in 37 C.F.R. §§ 1.821-1.822, and incorporated herein by reference. A dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co. p. 224).

Such substitutions are preferably made with those set forth as follows:

| | Conservative |
|---|---|
| Original residue | substitution(s) |
| Ala | Gly; Ser |
| Arg | Lys |
| Asn | Gln; His |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr, Ile |
| Phe | Met; Leu; Tyr |
| Ser | Thr |
| Thr | Ser |
| Trp | Tyr |
| Tyr | Trp; Phe |
| Val | Ile; Leu |

The term “analog(s)” as used herein refers to a composition that retains the same structure or function (e.g., binding to a receptor) as a polypeptide or nucleic acid herein. Examples of analogs include peptidomimetics, peptide nucleic acids, small and large organic or inorganic compounds, as well as derivatives and variants of a polypeptide or nucleic acid herein. The term “derivative” or “variant” as used herein refers to a peptide or nucleic acid that differs from the naturally occurring polypeptide or nucleic acid by one or more amino acid or nucleic acid deletions, additions, substitutions or side-chain modifications. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally-occurring amino acid of similar character either in relation to polarity, side chain functionality or size.

Substitutions encompassed by the present invention may also be “non-conservative”, in which an amino acid residue which is present in a peptide is substituted with an amino acid having different properties, such as naturally-occurring amino acid from a different group (e.g., substituting a charged or hydrophobic amino acid with alanine), or alternatively, in which a naturally-occurring amino acid is substituted with a non-conventional amino acid. Preferably, amino acid substitutions are conservative.

Amino acid substitutions are typically of single residues, but may be of multiple residues, either clustered or dispersed. Additions encompass the addition of one or more naturally occurring or non-conventional amino acid residues. Deletion encompasses the deletion of one or more amino acid residues.

As stated above peptide derivatives include peptides in which one or more of the amino acids has undergone side-chain modifications. Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal. The carboxyl group may be modified by carbodiimide activation via O-acylisourea formation followed by subsequent derivitisation, for example, to a corresponding amide. Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. Any modification of cysteine residues must not affect the ability of the peptide to form the necessary disulphide bonds. It is also possible to replace the sulphydryl groups of cysteine with selenium equivalents such that the peptide forms a diselenium bond in place of one or more of the disulphide bonds.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Proline residue may be modified by, for example, hydroxylation in the 4-position. Other derivatives contemplated by the present invention include a range of glycosylation variants from a completely unglycosylated molecule to a modified glycosylated molecule. Altered glycosylation patterns may result from expression of recombinant molecules in different host cells.

Additional derivatives include alterations that are caused by expression of the polypeptide in bacteria or other host system as well as through chemical modifications. Preferably, the derivatives retain the desired activity. For example, a derivative of T2 may be a truncated version of T2 that retains T2's ability to bind one of its naturally occurring receptors or to inhibit angiogenesis.

The term “antagonist” is used herein to refer to a molecule inhibiting a biological activity. Examples of antagonist molecules include but are not limited to antibodies, antisense nucleic acids, siRNA nucleic acids, and other binding agents.

The term “antibody” or “antibodies” as used herein includes polyclonal antibodies, monoclonal antibodies (mAbs), chimeric antibodies, anti-idiotypic (anti-Id) antibodies to antibodies that can be labeled in soluble or bound form, as well as fragments, regions or derivatives thereof (e.g., separate heavy chains, light chains, Fab, Fab′, F(ab′)2, Fabc, and Fv).

The term “effective amount” as used herein means that amount of composition necessary to achieve the indicated effect.

The terms “gene therapy” and “genetic therapy” refer to the transfer of heterologous nucleic acids to the certain cells, target cells, of a mammal, particularly a human, with a disorder or conditions for which such therapy is sought. The nucleic acid is introduced into the selected target cells in a manner such that the heterologous DNA is expressed and a therapeutic product encoded thereby is produced. Alternatively, the heterologous nucleic acids can in some manner mediate expression of a nucleic acid that encodes the therapeutic product, it can encode a product, such as a peptide or RNA that in some manner mediates, directly or indirectly, expression of a therapeutic product. Genetic therapy can also be used to nucleic acid encoding a gene product replace a defective gene or supplement a gene product produced by the mammal or the cell in which it is introduced. The introduced nucleic acid can encode a therapeutic compound, such as a growth factor inhibitor thereof, or a tumor necrosis factor or inhibitor thereof, such as a receptor thereof, that is not normally produced in the mammalian host or that is not produced in therapeutically effective amounts or at a therapeutically useful time. The heterologous DNA encoding the therapeutic product can be modified prior to introduction into the cells of the afflicted host in order to enhance or otherwise alter the product or expression thereof.

The term “homodimer” as used herein refers to two monomers that are complexed together either covalently or non-covalently wherein the two compounds are identical.

The term “homolog” or “homologous” as used herein refers to homology with respect to structure and/or function. With respect to sequence homology, sequences are homologs if they are at least 50%, preferably at least 60%, more preferably at least 70%, more preferably at least 80%, more preferably at least 90%, more preferably at least 95% identical, more preferably at least 97% identical, or more preferably at least 99% identical. The term “substantially homologous” refers to sequences that are at least 90%, more preferably at least 95% identical, more preferably at least 97% identical, or more preferably at least 99% identical. Homologous sequences can be the same functional gene in different species.

The term “host” as used herein refers to an organism that expresses a nucleic acid of this invention in at least one of its cells. The term “host cell” as used herein refers to a cell which expresses the nucleotide sequences according to this invention.

The term “inhibit” as used herein refers to prevention or any detectable reduction or elimination of a condition.

The term “isolated” as used herein refers to a compound or molecule (e.g., a polypeptide or a nucleic acid) that is relatively free of other compounds or molecules that it normally is associated with in vivo. In general, an isolated polypeptide constitutes at least about 75%, more preferably about 80%, more preferably about 85%, more preferably about 90%, more preferably about 95%, or more preferably about 99% by weight of a sample containing it.

The term “mini-TrpRS” as used herein refers to a polypeptide having amino acid sequence selected from the group consisting of SEQ ID NOS: 2, 3, 14, 17, 26, 29, 38, 41, 50, 53, and any homologs and analog thereof.

The term “multi-unit complex” as used herein refers to a complex of one or more monomer units that are complexed together covalently or non-covalently. Examples of multi-unit complexes include dimers, trimers, etc.

The term “nucleic acid” or “nucleic acid molecule” as used herein refers to an oligonucleotide sequence, polynucleotide sequence, including variants, homologs, fragments, or analogs thereof. A nucleic acid may include DNA, RNA, or a combination thereof. A nucleic acid may be naturally occurring or synthetic, double-stranded or single-stranded, sense or antisense strand.

As used herein the term “operably linked” wherein referring to a first nucleic acid sequence which is operably linked with a second nucleic acid sequence refers to a situation when the first nucleic acid sequence is placed in a functional relationship with the second nucleic acid sequence. For instance, a promoter is operably linked to a coding sequence if the promoter effects the transcription or expression of the coding sequence. Generally, operably linked nucleic acid sequences are contiguous and, where necessary to join two protein coding regions, the open reading frames are aligned.

The term “peptidomimetic” as used herein refers to both peptide and non-peptide agents that mimic aspects of a polypeptide. Non-hydrolyzable peptide analogs of critical residues can be generated using benzodiazepine (see Freidinger et al. in Peptides: Chemistry and Biology, G. R. Marshall ed., ESCOM Publisher: Leiden, Netherlands, 1988), azepine (see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), .beta.-turn dipeptide cores (Nagai et al. (1985) Tetrahedron Lett 26:647; and Sato et al. (1986) J Chem Soc Perkin Trans 1:1231), and beta.-aminoalcohols (Gordon et al. (1985) Biochem Biophys Res Commun 126:419; and Dann et al. (1986) Biochem Biophys Res Commun 134:71).

The term “polypeptide”, “peptide”, “oligopeptides” or “protein” refers to any composition that includes two or more amino acids joined together by a peptide bond. It will be appreciated that polypeptides often contain amino acids other than the 20 amino acids commonly referred to as the 20 naturally occurring amino acids, and that many amino acids,-including the terminal amino acids, may be modified in a given polypeptide, either by natural processes such as glycosylation and other post-translational modifications, or by chemical modification techniques which are well known in the art.

Among the known modifications which may be present in polypeptides of the present invention include, but are not limited to, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The term “receptor” refers to a biologically active molecule that specifically binds to (or with) other molecules. The term “receptor protein” can be used to more specifically indicate the proteinaceous nature of a specific receptor. For example, the term “T2 receptor” refers to a biologically active molecule that specifically binds to (or with) T2.

The term “T1” refers to a polypeptide having an amino acid sequence selected from the group consisting of SEQ ID NOS: 4, 5, 13, 16, 25, 28, 37, 40, 49, 52, and any homologs and analogs thereof.

The term “T2” refers to a polypeptide having an amino acid sequence selected from the group consisting of SEQ ID NOS: 6, 7, 12, 15, 24, 27, 36, 39, 48, 51, and any homolog and analog thereof.

The term “treating” as used herein refers to eliminating, reducing, or alleviating symptoms in a subject, or preventing symptoms from occurring, worsening, or progressing.

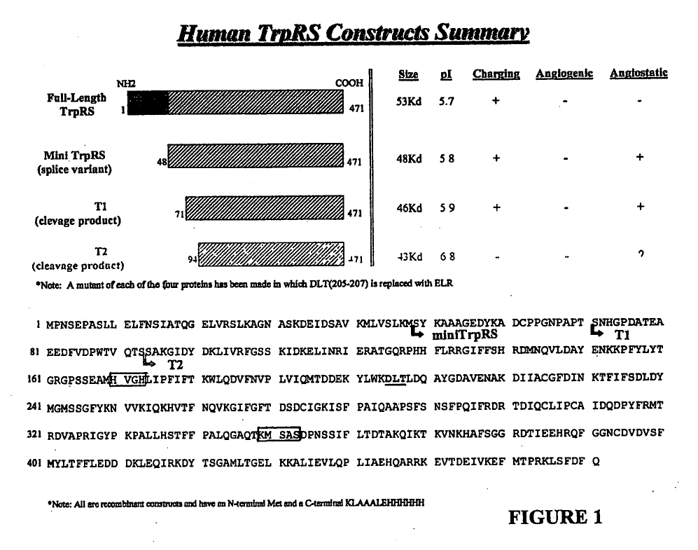

The term “TrpRS” or “tryptophanyl tRNA synthetase” as used herein refers to the full length tryptophanyl-tRNA synthetase as illustrated in FIG. 1, wherein amino acid residues 213 is either Gly or Ser and amino acid residue 214 is either Asp or Tyr (independently of the other). Thus, the terms “GD variant” “SD variant” “GY variant” and “SY variant” as used herein refer to TrpRS or fragment thereof with the corresponding amino acid residues in the above location within the polypeptide.

The term “tRS” as used herein means a tRNA synthetase polypeptide and/or nucleic acids encoding such polypeptide, whether naturally occurring or non-naturally occurring.

The term “truncated tRNA synthetase polypeptides” means polypeptides that are shorter than the corresponding full length tRNA synthetase.

II. Compositions

Aminoacyl-tRNA synthetases (tRS) are ancient proteins that are essential for decoding genetic information during the process of translation. There are two classes of tRS. The first class, class I, contains a common loop with the signature sequence KMSKS (and HIGH, as part of a Rossman dinucletide binding fold of parallel beta sheets (“Rossman fold domain”). (Sever et al., Biochem. 35, 32-40 (1996)). The second class, Class II, have an entirely different topology of dinucleotide binding bases on antiparaellal beta sheets.

Tryptophanyl-tRNA synthetase (TrpRS) is a Class I tRS. It is believed that expression of TrpRS is stimulated by interferon (“IFN”) (e.g, IFN-gamma) and/or tumor necrosis factor (“TNF”) (e.g., TNF-alpha). IFN-gamma is responsible for antiviral and anti-proliferative state of animal cells. See Kisselev, L., Biochimie 75, 1027-1039 (1993). Stimulation of TrpRS by IFN occurs at the transcriptional level by a consensus regulatory sequence designated IFN-stimulated response element (“ISRE”). An examination of ISRE sequences from a number of IFN-response genes indicates a common motif of GGAAAN(N/−)GAAA. Thus the present invention contemplates the use of the compositions herein to treat IFN and/or TNF mediated conditions, and in particular IFN-gamma and/or TNF-alpha mediated conditions.

Mammalian TrpRS molecules have an amino-terminal appended domain. In normal human cells, there are two forms of TrpRS that can be detected: a major form consisting of the full-length molecule (amino acid residues 1-471 of SEQ ID NO: 1) and a minor truncated form (“mini TrpRS”; a polypeptide comprising amino acid sequence SEQ ID NOS: 3, 14, 19, or 20). In any of the Trp-RS embodiments herein amino acids 213 can be either a Gly or Ser and amino acid 214 can be either an Asp or Tyr. Such variants may be referred to herein as the GD variant, GY variant, SD variant and SY variant.

The minor form is generated by the deletion of the amino-terminal domain through alternative splicing of the pre-mRNA (Tolstrup et al., J. Biol. Chem. 270:397-403 (1995)). The amino-terminus of mini TrpRS has been determined to be the methionine residue at position 48 of the full-length TrpRS molecule. Alternatively, truncated TrpRS can be generated by proteolysis. Lemaire et al., Eur. J. Biochem. 51:237-52 (1975). For example, bovine TrpRS is highly expressed in the pancreas and is secreted into the pancreatic juice (Kisselev, Biochimie 75:1027-39 (1993)), thus resulting in the production of a truncated TrpRS molecule. These results suggest that truncated TrpRS can have a function other than the aminoacylation of tRNA.

Studies indicate that the full-length TrpRS does not inhibit angiogenesis, whereas mini-TrpRS inhibits VEGF-induced cell proliferation and migration (Wakasugi et al., Proc. Natl. Acad. Sci. 99: 173-177 (2002)). In particular, a chick CAM assay shows that mini TrpRS blocks angiogenic activity of VEGF. Thus, removal of the first 48 amino acid residues exposes the anti-angiogenic activity of TrpRS. TrpRS and mini-TrpRS are further described in International Application Nos. PCT/US01/08966 and PCT/US01/8975, both filed Mar. 21, 2001, the disclosures of which are incorporated herein by reference in their entirety.

Additional fragments of TrpRS that have angiostatic activity are referred to herein as T1 and T2. Treatment of TrpRS with PMN elastase results in two additional products: a 47 kDa fragment (super mini-TrpRS or T1; e.g., SEQ ID NO: 13, 16, 25, 28, 37, 40, 49, and 52) and an approximately 43 kDa fragment (T2-TrpRS or T2; e.g., SEQ ID N: 12, 15, 24, 27, 36, 39, 48, and 51). Terminal amino acid analysis has revealed Ser-71 and Ser-94, respectively, as the NH₂-terminal residues for these fragments. Both T1 and T2 have been shown to be potent antagonists of in vivo angiogenesis as illustrated in the examples below. T1 and T2 are further described in U.S. Provisional Application No. 60/270,951 filed on Feb. 23, 2001, for “Tryptophanyl-tRNA Synthetase Derived Polypeptides Useful for the Regulation of Angiogenesis” as well as U.S. patent application Ser. No. 10/080,839, filed Feb. 22, 2002, and International Application No. PCT/US02/05185, filed Feb. 22, 2002, the disclosures of which are incorporated herein by reference in their entirety. Methods for preparing T2 are further disclosed in U.S. Provisional Application No. 60/598,019, filed Aug. 8, 2004, entitled “Composition of and Purification Methods for Low-Endotoxin Therapeutic Agents”, which is incorporated herein by reference in its entirety.

1. Polypeptides

The present invention relates to compositions comprising a tRNA synthetase fragment having angiogenic or angiostatic (anti-angiogenic) activity. Examples of tRNA synthetase fragments of the present invention include tryptophanyl tRNA synthetase fragments and tyrosyl tRNA synthetase fragments. Such fragments are preferably mammalian, or more preferably human.

In some embodiments, a composition of the present invention comprises a tRNA synthetase fragment, wherein the tRNA synthetase fragment comprises, consists essentially of, or alternatively consists of an amino acid sequence selected from the group of SEQ ID NO: 12, 15, 24, 27, 36, 39, 48, 51, and any homologs and analogs thereof. Preferably, such tRNA synthetase fragment is less than 45 kD, more preferably less than 44 kD, 43.9 kD, 43.8 kD, 43.7 kD, 43.6 kD, or more preferably less than 43.5 kD. Preferably such fragments are anti-angiogenic.

In some embodiments, a composition of the present invention comprises a tRNA synthetase fragment, wherein the tRNA synthetase fragment comprises, consists essentially of, or alternatively consists of an amino acid sequence selected from the group of SEQ ID NO: 13, 16, 25, 28, 37, 40, 49, 52, and any homologs and analogs thereof. Preferably, such tRNA synthetase fragment is less than 48 kD, more preferably less than 47 kD, or more preferably less than 46 kD. Preferably such tRNA synthetase fragment is anti-angiogenic.

In some embodiments, a composition of the present invention comprises a tRNA synthetase fragment, wherein the tRNA synthetase fragment comprises, consists essentially of, or alternatively consists of an amino acid sequence selected from the group of SEQ ID NO: 14, 17, 26, 29, 38, 41, 50, 53, and any homologs and analogs thereof. Preferably, such tRNA synthetase fragment is less than 53 kD, more preferably less than 52 kD, more preferably less than 51 kD, more preferably less than 50 kD, or more preferably less than 49 kD. Preferably such tRNA synthetase fragment is anti-angiogenic.

In any of the embodiments herein, a tRNA synthetase fragment is preferably isolated. Moreover, in any of the embodiments herein, a tRNA synthetase fragment is preferably purified. Methods for purifying a tRNA synthetase fragment are described in U.S. Provisional Application No. 60/598,019, which is incorporated herein by reference for all purposes.

In some embodiments, a tRNA synthetase fragment of the invention has an isoelectric point (pI) of less than 10.0, more preferably less than 9.0, or more preferably less than 8.0. In some embodiments, a tRNA synthetase fragment has an isoelectric point of 5.0 to 9.0, more preferably 6.0 to 8.0, or more preferably 7.4 to 7.8. In some embodiments, a tRNA synthetase fragment of the invention has a pI greater than 7.1, 7.2, 7.3, 7.4, 7.5, 7.6, 7.7, 7.8, or 7.9. Preferably, a tRNA synthetase fragment of the invention has a pI of about 7.6. In some embodiments, a tRNA synthetase fragment herein has a hydrophobic cleft.

Such fragments may form monomers of a multi-unit complex. A multi-unit complex of the present invention can include, for example, at least 2, 3, 4, or 5 monomers. Both the monomer and multi-unit complexes of the present invention may be soluble and may be isolated or purified to homogeneity.

Examples of naturally occurring or synthetic polypeptides of the present invention having angiostatic activity include those having an amino acid sequence selected from the group consisting of SEQ ID NOS: 12-17, 24-29, 36-41, 48-53, and any homologs and analogs thereof. In addition, fragments of the above polypeptides having angiostatic activity are also contemplated by the present invention.

In some embodiments, a tRNA synthetase fragment is part of a multi-unit complex. A multi-unit complex of the invention can be, for example, a dimer or trimer. A multi-unit complex of the invention comprises of at least two monomer units that are associated with each other. A monomers unit within a multi-unit complex can be associated to one another monomer unit either covalently, non-covalently, or both covalently and non-covalently.

Monomer units a multi-unit complex may be different, homologous, substantially homologous, or identical to one another. However, a multi-unit complex of the invention includes at least one monomer comprising a TRNA synthetase fragment, or more preferably at least two monomers comprising a tRNA synthetase fragment. For example, the present invention contemplates a dimer composition, wherein each monomer unit of the dimer is selected from the group consisting of mini-TrpRS, T1, and T2. In some embodiments, such dimer compositions are isolated. In some embodiments, such dimer compositions are soluble. In some embodiments, such dimers are homodimers.

Covalently linked monomers can be linked directly (by bonds) or indirect (e.g., via a linker). For directly linking the polypeptide monomers herein, it may be beneficial to modify the polypeptides herein to enhance dimerization. For example, one or more amino acid residues of a tRNA synthetase fragment may be modified by the addition or substation by one or more cysteines. A tRNA synthetase fragment modified under the present invention is preferably a tryptophanyl tRNA synthetase fragment. Such fragments are preferably mammalian, or more preferably human. Such fragments have angiostatic activity and are preferably selected from the group consisting of SEQ ID NOS: 12-17, 24-29, 36-41, 48-53, and any homologs and analogs thereof. Methods for creating cysteine substitutions, such as by site directed mutagenesis, are known to those skilled in the art.

Preferably, such modification occurs in the dimerization domain of the fragment. A dimerization domain refers to that domain which forms covalent and/or non-covalent bonds with a second monomer. For example, the dimerization domain of full length Trp-RS (SEQ ID NO: 1) is between amino acid residues about 230 to about 300, or more preferably between amino acid residues about 237 to about 292. In another example, the dimerization domain for a polypeptide of SEQ ID NO: 13, a T1, is between amino acid residues about 160 to about 230, or more preferably between amino acid residues about 167 to about 222. In another example, the dimerization domain for a polypeptide of SEQ ID NO: 12, a T2, is between amino acid residues about 137 to about 157, or more preferably between amino acid residues about 144 to about 149. For other angiogenic fragments of a tRNA synthetase, the dimerization region may be any region that is homologous to the above regions or SEQ ID NO: 60.

The addition or substitution of cysteines can create disulfide bridges, linking two or more monomers covalently. Preferably, two or more of the modified polypeptide herein are covalently linked to form a multi-unit (monomer) complex. A multi-unit complex comprises of at least two, three, four, or five monomers. The various monomers in a multi-unit complex may be different, homologous, substantially homologous, or identical to one another. In preferred embodiments, two or more of the various monomers in a multi-unit complex are substantially homologous to one another or identical to one another.

A linker of the present invention is preferably long enough to allow the two dimers to align in the head-to-tail orientation (N-terminus to C-terminus). In some embodiments, a linker is at least about 3, more preferably about 30, more preferably about 150, more preferably about 300, or more preferably about 450 atoms in length. Linker sequences, which are generally between 2 and 25 amino acids in length, are well known in the art and include, but are not limited to, the glycine(4)-serine spacer (GGGGS x3) described by Chaudhary et al. (1989). These and other linkers can be used in the present invention.

Examples of non-covalent bonds (associations) include electrostatic bonds, ionic bonds, hydrogen bonds, Van der Waals bonds, and hydrophobic effect.

In any one of the embodiments herein, a polypeptide can be any of the above wherein (i) one or more of the amino acid residues are substituted with a conserved or non-conserved amino acid residue (preferably a conserved amino acid residue) and such substituted amino acid residue is or is not be one encoded by the genetic code; (ii) one or more of the amino acid residues includes a substituent group; (iii) the polypeptide is fused with another compound, (e.g., a compound to increase the half-life of the polypeptide or target it to a specific receptor, cell, tissue, or organelle), (iv) additional amino acids are fused to the polypeptide, such as a leader or secretory sequence or a sequence which is employed for purification of the polypeptide or a proprotein sequence; or (v) one or more of the amino acid residues are substituted with a non-conserved amino acid residue (preferably cysteine) and such substituted amino acid residue form a disulfide bridge with a second polypeptide (e.g., to form a dimer or homodimer). Such derivatives are deemed to be within the scope of those skilled in the art from the teachings herein.

For example, any of the polypeptides herein can be modified to improve stability and increase potency by means known in the art. For example, L-amino acids can be replaced by D-amino acids, the amino terminus can be acetylated, or the carboxyl terminus modified, e.g., ethylamine-capped (Dawson, D. W., et al., Mol. Pharmacol., 55: 332-338 (1999)) or glycosylated.

In another example, the polypeptides herein can be fused to another protein or portion thereof. For example, mini-TrpRS, T1 or T2 polypeptide or portion thereof, can be operably linked to another polypeptide moiety to enhance solubility. Examples of a protein which can be fused with mini-TrpRS, T1 or T2 or portions thereof to enhance solubility include a plasma protein or fragment thereof. In other embodiments, mini-TrpRS, T1 or T2 polypeptide or portion thereof, can be operably linked to another polypeptide moiety to target the molecule to a specific tissue or cell type. For example, mini-TrpRS, T1 or T2 polypeptides or portions thereof, can be operable linked to an antibody that specifically binds the photoreceptor cells in the eye, a particular tumor cell, or a particular organelle. In some embodiments, mini-TrpRS, T1 or T2 polypeptide may be operably linked to a polypeptide moiety that helps reduce immune response, for example, a constant F(c) region of an immunoglobulin.

In another embodiment, the polypeptides herein include a leader sequence. A leader sequence can be used to allow the polypeptide to enter into a specific cell. Thus, the present invention contemplates a polypeptide of SEQ ID NOS: 12-17, 24-29, 36-41, and 58-53.

In another example, the polypeptides herein can be modified for enhanced dimerization. Modifications that enhance dimerization of a polypeptide include alternations (e.g., substitutions or additions) to the naturally occurring sequence which enhance covalent and/or non-covalent interactions of the polypeptide with another monomer. Preferably modifications are made within a dimerization domain.

For tryptophanyl-tRNA synthetase and fragments thereof, the dimerization domain is approximately between amino acid residues 230 and 300, or more preferably approximately between amino acid residues 237 and 292 of the full length Trp-tRS (SEQ ID NO: 1). Such polypeptides (preferably mini-TrpRS, T1, and T2) have enhanced dimerization capabilities. Thus, in some embodiments, the present invention contemplates a mini-TrpRS monomer with a cysteine addition or substitution approximately between amino acid residues 183 and 253, or more preferably approximately between amino acid residues 190 and 245. In some embodiments, the present invention contemplates a T1 monomer with a cysteine addition or substitution approximately between amino acid residues 160 and 230, or more preferably between amino acid residues 167 and 222. In some embodiments, the present invention contemplates a T2 monomer with a cysteine addition or substitution approximately between amino acid residue 137 and 208, or more preferably between amino acid residue 144 and 200.

It is further contemplated by the present invention that any of the cysteine modified polypeptides may dimerize to form tRNA synthetase dimers. In preferred embodiments, such dimerization occurs naturally as a result of expressing any of the above polypeptide(s) using a vector that encodes a single tRNA synthetase fragment, and allowing such expressed fragments to naturally dimerize.

In some embodiments, the present invention contemplates a composition comprising a first tRNA synthetase fragment and a second tRNA synthetase fragment, wherein the first tRNA synthetase fragment has a methionine at its N-terminus (“Met-Trp-RS fragment”) and wherein the second tRNA synthetase does not have a methionine at its N-terminus (“non-Met-Trp-RS fragment”). Preferably, the tRNA synthetase fragments herein are tryptophanyl-tRNA synthetase fragments. Examples of Trp-tRNA synthetase fragments having a methionine on their N-terminus include those of SEQ ID NOS: 15-17, 27-29, 39-41, 51-53, and homologs and analogs thereof. Examples of Trp-tRNA synthetase fragments that do not have a methionine on their N-terminus include those if of SEQ ID NOS: 12-14, 24-26, 36-38, 48-50, and homologs and analogs thereof. All other angiostatic fragments of Trp-tRNA synthetase are contemplated herein. The methionine may be synthetically added to their N-terminus.

In some embodiments, more than 50% of a composition of a Trp-tRNA synthetase fragment has methionine at its N-terminus. In other embodiments, more than 50% of a composition of a Trp-tRNA synthetase fragment does not have a methionine at its N-terminus.

Any of the above compositions can further comprise a therapeutic agent, such as an antineoplastic agent, an anti-inflammatory agent, an antibacterial agent, an angiogenic agent, an antiviral agent, and an anti-angiogenic agent. Examples of such agents are disclosed herein. Preferably, the therapeutic agent is an anti-angiogenic agent and is either a VEGF antagonist or an integrin antagonist.

2. Antibodies

In another aspect, the invention provides a peptide comprising or consisting of an epitope-bearing portion of the polypeptides described herein. The term “epitope” as used herein, refers to a portion of a polypeptide having antigenic or immunogenic activity in an animal, preferably a mammal, and most preferably in a human. Antigenic epitope-bearing peptides of the polypeptides of the invention are useful to raise antibodies, including monoclonal antibodies that bind specifically to a polypeptide of the invention. The term “antigenic epitope,” as used herein, is defined as a portion of a protein to which an antibody can immunospecifically bind its antigen as determined by any method well known in the art, for example, by the immunoassays

Antigenic epitope-bearing polypeptides of the invention preferably contain a sequence of at least about five or about seven, more preferably at least about nine or about eleven amino acids, and more preferably between at least about to about 0 or more preferably between about 10 to about 20 amino acids contained within a tRNA synthetase fragment, or more preferably a tryptophanyl tRNA synthetase fragment. Such fragments are preferably mammalian, or more preferably human. The tRNA fragments herein have angiostatic activity. Examples of human tryptophanyl tRNA synthetase fragments with angiostatic activity include, but are not limited to SEQ ID NOS: 12-17, 24-29, 36-41, 48-53, and homologs and analogs thereof. In this context “about” includes the particularly recited value and values larger or smaller by several (5, 4, 3, 2, or 1) amino acids.

In some embodiments, such epitope-bearing polypeptides are “N-terminus epitopes.” The phrase “N-terminus epitopes” as used herein refer to a peptide having an amino acid sequence that is closer to the N-terminus than the C-terminus of a polypeptide of the invention (e.g., SEQ ID NOS: 12-17, 24-29, 36-41, and 48-53). In some embodiments, such epitope-bearing polypeptides comprise or consist of the N-terminus of a polypeptide of the invention (e.g., SEQ ID NOS: 12-17, 24-29, 36-41, and 48-53).

Examples of such epitope-bearing polypeptides include polypeptide comprising, or alternatively consisting of: amino acid residues of about 1 to about 5, about 1 to about 15, or about 1 to about 25 of SEQ ID NOS: 12-17, 24-29, 36-41, 48-53, and any homologs or analogs thereof; amino acid residues of about 10 to about 15, about 10 to about 25, or about 10 to about 35 of SEQ ID NOS: 12-17, 24-29, 36-41, 48-53, and any homologs or analogs thereof; amino acid residues of about 20 to about 25, about 20 to about 35, or about 20 to about 45 of SEQ ID NOS: 12-17, 24-29, 36-41, 48-53 and any homologs and analogs thereof.

The above polypeptides can be used for research purposes (e.g., to distinguish between one fragment and another), for diagnostic purposes (e.g., to identify and quantify angiogenic/angiostatic fragments); and/or for therapeutic purposes (e.g., to inhibit angiostatic activity of an angiostatic tRNA synthetase fragment).

For example, in some embodiments, antibodies of the present invention can distinguish between any two of the following mini-TrpRS, T1, and T2. In some embodiments, antibodies of the present invention can distinguish between a tRNA synthetase fragment having and not having a methionine in its N-terminus. (For example, an antibody can distinguish between SEQ ID NOS: 12 and 15; or between SEQ ID NOS: 13 and 16; or between SEQ ID NOS: 14 and 17; or variants thereof.) In some embodiments, antibodies of the present invention can distinguish between two variants of a tRNA synthetase fragment. (For example, an antibody of the present invention may distinguish between two polypeptide selected from the following group: SEQ ID NOS: 12, 24, 36, and 48.)

Other antibodies that bind the dimerization domain or receptor binding domain may also be useful as therapeutics to treat or prevent a condition associated with diminished vascular growth (an anti-angiogenic condition).

Moreover, calibration of the amount of tRNA fragments that are angiogenic and/or non-angiogenic may permit the diagnosis of angiogenesis-mediated condition.

Polynucleotides encoding these antigenic epitope-bearing peptides are also encompassed by the present invention.

Epitope-bearing polypeptides of the present invention may be used to induce antibodies according to methods well known in the art including, but not limited to, in vivo immunization, in vitro immunization, and phage display methods.

If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde.

For making a polyclonal antibody, animals such as, for example, rabbits, rats, and mice are immunized with either free or carrier-coupled peptides, for instance, by intraperitoneal and/or intradermal injection of emulsions containing about 100 micrograms of an epitope-bearing peptide and possibly a carrier protein and Freund's adjuvant or any other adjuvant known for stimulating an immune response. Several booster injections may be needed, for instance, at intervals of about two weeks, to provide a useful titer of anti-peptide antibody that can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

More preferably, the present invention contemplates monoclonal antibodies that are able to specifically bind to one or more of the polypeptides herein. Monoclonal antibodies can be readily prepared through use of well-known techniques such as those exemplified in U.S. Pat. No. 4,196,265, which is incorporated herein by reference for all purposes. Typically, a technique involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, for example, by the addition of agents that block the de novo synthesis of nucleotides in the tissue culture media. This culturing provides a population of hybridomas from which specific hybridomas are selected. Typically, selection of hybridomas is performed by culturing the cells by single-clone dilution in microtiter plates, followed by testing the individual clonal supernatants for reactivity with antigen-polypeptides. The selected clones can then be propagated indefinitely to provide the monoclonal antibody. Preferably, a monoclonal antibody of the present invention is also humanized.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo.

The present invention also contemplates fragment, regions or derivatives of the above antibodies. Such fragments include separate heavy chains, light chains, Fab, Fab′, F(ab′)2, Fabc, and Fv.

3. Nucleic Acids

The present invention also contemplates polynucleotide sequences encoding any of the polypeptides herein. In some embodiments, a polynucleotide sequence encodes two or more of the polypeptides herein. Preferably, the polynucleotide sequences of the present invention are isolated.

For example, the present invention contemplates polynucleotide sequences that encode one or more, or two or more tRNA synthetase fragments. The tRNA synthetase fragments can be fragments of any one or more of the tRNA synthetases known in the art, but more preferably either of a tryptophanyl tRNA synthetase or a tyrosynyl tRNA synthetase. A tRNA synthetase of the present invention is preferably mammalian, or more preferably human. Furthermore, fragments of such tRNA synthetases preferably have angiostatic activity.

For example, in some embodiments, a polynucleotide sequence of the present invention encodes one or more angiostatic fragments of a tRNA synthetase. Examples of angiostatic fragments of a tryptophanyl tRNA synthetase include mini-TrpRS, T1, and T2 and any angiostatic fragments, homologs or analogs thereof. Thus, in some embodiments, a polynucleotide of the present invention encodes a tryptophanyl tRNA synthetase fragment selected from the group consisting of SEQ ID NOS: 12-17, 24-29, 36-41, 48-53 and any homologs and analogs thereof. Examples of polynucleotide sequences encoding such fragments are the polynucleotide sequence of SEQ ID NOS: 18-23, 30-35, 42-47, and 54-59. As the DNA code is degenerative, such that more than one codon can encode a single amino acid residue, the above polynucleotide sequences are exemplary and not intended to be limiting in any way.

In some embodiments, a polynucleotide sequence of the present invention encodes two or more of the polypeptides herein. For example, a polynucleotide of the present invention can encode a first tRNA synthetase fragment and a second tRNA synthetase fragment. The first tRNA synthetase fragment can be selected from the group consisting of SEQ ID NOS: 12-17, 24-29,36-41, 48-53, and any homologs and analogs thereof. The second tRNA synthetase fragment can also be selected from the group consisting of SEQ ID NOS: 12-17, 24-29, 36-41, 48-53, and any homologs and analogs thereof. The first and the second tRNA synthetase fragments can be different, homologous, substantially homologous, or identical.

In some embodiments, the nucleotide sequences encoding two or more copies of a polypeptide sequence can be fused in tandem. When two nucleotide sequences encoding polypeptides are fused in tandem each polypeptide can have its own orientation such that when the two nucleotide sequences are expressed the encoded polypeptides can result in a C—N,N—N, C—C, or C—N terminal connection. In preferred embodiments, expression of the nucleotide sequences herein result in the N terminus of the second polypeptide being covalently linked to the C-terminus of the first polypeptide.

In some embodiments, a polynucleotide sequence encoding two or more tRNA synthetase fragments may also encode a linker. A nucleotide sequence encoding a linker can be inserted between two nucleotide sequences tRNA synthetase fragments. A nucleotide sequence encoding a linker can be long enough to allow a first tRNA synthetase fragment and a second tRNA synthetase fragments to freely rotate and dimerze with one another. In some embodiments, a nucleotide sequence encoding a linker is preferably at least 9, more preferably at least 30, more preferably at least around 60, or more preferably at least around 90-nucleotides in length.

In some embodiments, a polynucleotide sequence encoding a first tRNA synthetase fragment can be inserted within a polynucleotide sequence encoding a second tRNA synthetase fragment. This will result in translation of a first segment of the first tRNA synthetase fragment, the complete translation of the second tRNA synthetase fragment, and then translation of the remaining segment of the first tRNA synthetase fragment.

In some embodiments, a polynucleotide sequence herein encodes a modified tRNA synthetase fragment. An example of a modified tRNA synthetase fragment is one wherein the fragment has been modified (e.g., by addition or substitution of amino acids) to insert one or more non-naturally occurring cysteines into the fragment. Preferably, the tRNA synthetase fragment is a tryptophanyl tRNA synthetase fragment, or more preferably a fragment selected from the group consisting of SEQ ID NOS: 12-17, 24-29, 36-41, 48-53, and any homologs or analogs thereof.

Preferably, non-naturally occurring cysteine(s) are inserted (e.g., by addition or substitution) into the dimerization domain of the fragment. The insertion of such a cysteine can be made at the nucleic acid level using recombinant technology. Nucleic acid sequences that can be modified by the following invention to include cysteines include, but are not limited to, SEQ ID NOS: 18-23, 30-35, 42-47, 54-59, and any homologs, and analogs thereof.

In some embodiments, a polynucleotide of the invention encodes two or more modified tRNA synthetase fragments. For example, a polynucleotide of the present invention can encode 2 or more tryptophanyl tRNA synthetase fragments wherein each fragment is modified to include at least one non-naturally occurring cysteine in its dimerization domain. Examples of tryptophanyl TRNA synthetase fragments that can be modified as follows include, but are not limited to SEQ ID NOS: 12-17, 24-29, 36-41, 48-53, and any homologs or analogs thereof.

Any of the polynucleotides herein are preferably fused in the same reading frame to a polynucleotide sequence which aids in expression and secretion of a polypeptide from a host cell. This results in an expression vector. An expression vector can be used to express the polynucleotides in a host cell.

In some embodiments, a leader sequence which functions as a secretory sequence for controlling transport of a polypeptide from the cell can be fused after the open reading frame sequence. A polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains. Thus, for example, the polynucleotide of the present invention can encode for a mature protein, or for a protein having a prosequence or for a protein having both a prosequence and presequence (leader sequence). Preferably, when a polynucleotide sequence of the present invention encodes a prosequence, such prosequence is cleaved in the vitreous of the eye or at a target cancer cell or tumor.

In some embodiments, the pre or pro sequences encode for antibodies or antibody fragments that bind to a target cell (e.g., photoreceptors). Again, the pre or pro sequence can include a protease cleavage site that will allow for the sequence to be automatically cleaved upon reaching its desired site, thus activating the compositions herein.

The polynucleotides of the present invention can also have the coding sequence fused in frame to a marker sequence which allows for purification of the polypeptide of the present invention. The marker sequence can be a hexa-histidine tag supplied by a pQE-9 vector to provide for purification of the mature polypeptide fused to the marker in the case of a bacterial host, or, for example, the marker sequence can be a hemagglutinin (HA) tag when a mammalian host, e.g. COS-7 cells, is used. The HA tag corresponds to an epitope derived from the influenza hemagglutinin protein (Wilson, I., et al., Cell, 37:767 (1984)).

The present invention further relates to polynucleotides that hybridize to any of the sequences described herein, preferably under stringent conditions. A stringent condition refers to a condition that allows nucleic acid duplexes to be distinguished based on their degree of mismatch. Such polynucleotides (e.g., antisense and RNAi) can be used to inhibit the expression of an angiostatic tRNA fragment or angiogenic tRNA fragment depending upon the desired outcome. Such polynucleotides can also serve as probes and primers for research and diagnostic purposes.

Antisense nucleic acids are nucleotide sequences which are complementary to the coding strand of a double-stranded cDNA molecule or to an mRNA sequence of a target nucleotide sequence, preferably encoding a positive angiogenesis factor, e.g., VEGF. Antisense nucleic acids can be used as an agent to inhibit angiogenesis in the methods described herein. It inhibits translation by forming hydrogen bonds with a sense nucleic acid. Antisense nucleic acid can be complementary to an entire angiogenic coding region (e.g., VEGF) or only to a portion thereof.

An antisense oligonucleotide herein can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

In some embodiments, double stranded nucleic acids can be used to silence genes associated with angiogenesis (e.g., tryptophanyl tRNA synthetase and/or tyrosyl tRNA synthetase) by RNA interference. RNA interference (“RNAi”) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al. Nature May 24, 2001; 411(6836):494-8).

In one embodiment, transfection of small (less than 50, more preferably 40, more preferably 30 or more preferably 20 nt) dsRNA specifically inhibits gene expression (reviewed in Caplen (2002) Trends in Biotechnology 20:49-51). Briefly, RNAi is thought to work as follows. dsRNA corresponding to a portion of a gene to be silenced is introduced into a cell. The dsRNA is digested into small dsRNA nucleotide siRNAs, or short interfering RNAs. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA at about 12 nucleotides from the 3′ terminus of the siRNA (reviewed in Sharp et al (2001) Genes Dev 15: 485-490; and Hammond et al. (2001) Nature Rev Gen 2: 110-119).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem Biophys Res Commun Mar. 2, 2001; 281(3):639-44), providing yet another strategy for gene silencing. In some embodiments, the present invention relates to methods of modulating angiogenesis by contacting a cell or tissue with an RNAi or antisense complementary to a tRNA synthetase (e.g., TyrRS or TrpRS) or a fragment thereof. For example an antisense or RNAi of the present invention can be complementary to a polynucleotide sequence selected from the group consisting of SEQ ID NOS: 18-23, 30-35, 42-47, 54-60, and any homologs and analogs thereof.

The polynucleotides of the present invention are preferably provided in an isolated form, and preferably are purified to homogeneity.

4. Vectors

The present invention also includes vectors (preferably expression vectors) which include polynucleotides of the present invention, host cells which are genetically engineered with vectors of the invention and the production of polypeptides of the invention by recombinant techniques.

The vectors of the present invention can be constructed using standard recombinant techniques widely available to one skilled in the art. Such techniques can be found in common molecular biology references such as Sambrook, et al., Molecular Cloning: A Laboratory Manual, Cold Spring Harbor Laboratory Press (1989), D. Goeddel, ed., Gene Expression Technology, Methods in Enzymology series, Vol. 185, Academic Press, San Diego, Calif. (1991), and Innis, et al. PCR Protocols: A Guide to Methods and Applications Academic Press, San Diego, Calif. (1990).

In preferred embodiments, the present invention contemplates recombinant construction of a vector which comprises one or more, or more preferably two or more, of the polynucleotide sequences described above. The constructs comprise a vector, such as a plasmid or viral vector, into which one or more, or more preferably two or more, polynucleotide sequence of the invention are inserted, in a forward or reverse orientation. Preferably, two polynucleotide sequences are inserted into a vector in tandem. The polynucleotide sequences can be adjacent to one another or separated by a linker.

5. Host Cells

Host cells of the invention are cells that express the nucleotide sequences described herein. Representative examples of appropriate hosts include bacterial cells, such as E. coli, Salmonella typhimurium, Streptomyces; fungal cells, such as yeast; insect cells, such as Drosophila and Sf9; animal cells such as CHO, COS or Bowes melanoma; plant cells, etc. The selection of an appropriate host is deemed to be within the scope of those skilled in the art from the teachings herein.

There are available to one skilled in the art multiple viral and non-viral methods suitable for introduction such nucleotide sequences into a target host cell.

Viral transduction methods can comprise the use of a recombinant DNA or an RNA virus comprising a nucleic acid sequence that drives or inhibits expression of a protein having sialyltransferase activity to infect a target cell. A suitable DNA virus for use in the present invention includes but is not limited to an adenovirus (Ad), adeno-associated virus (AAV), herpes virus, vaccinia virus or a polio virus. A suitable RNA virus for use in the present invention includes but is not limited to a retrovirus or Sindbis virus. It is to be understood by those skilled in the art that several such DNA and RNA viruses exist that can be suitable for use in the present invention.

“Non-viral” delivery techniques that have been used or proposed for gene therapy include DNA-ligand complexes, adenovirus-ligand-DNA complexes, direct injection of DNA, CaPO₄ precipitation, gene gun techniques, electroporation, liposomes and lipofection. Any of these methods are widely available to one skilled in the art and would be suitable for use in the present invention. Other suitable methods are available to one skilled in the art, and it is to be understood that the present invention can be accomplished using any of the available methods of transfection. Several such methodologies have been utilized by those skilled in the art with varying success. Lipofection can be accomplished by encapsulating an isolated DNA molecule within a liposomal particle and contacting the liposomal particle with the cell membrane of the target cell. Liposomes are self-assembling, colloidal particles in which a lipid bilayer, composed of amphiphilic molecules such as phosphatidyl serine or phosphatidyl choline, encapsulates a portion of the surrounding media such that the lipid bilayer surrounds a hydrophilic interior. Unilammellar or multilammellar liposomes can be constructed such that the interior contains a desired chemical, drug, or, as in the instant invention, an isolated DNA molecule.

a. Expression

Expression vectors can be used to express the polynucleotides herein in host cells. Expression vectors contain the appropriate polynucleotide sequences, such as those described herein, as well as an appropriate promoter or control sequence, can be employed to transform an appropriate host to permit the host to express the protein. Preferably an expression vector of the present invention expresses a polypeptide selected from the group consisting of SEQ ID NOS: 12-17, 24-29, 36-41,48-53, and any homologs and analogs thereof. A composition of the present invention may therefore be produced by transfecting a host cell with an expression vector or polynucleotide sequence that encodes a polypeptide comprising of, consisting essentially or, or consisting of an amino acid sequence SEQ ID NOS: 12-17, 24-29, 36-41, 48-53, or any homologs or analogs thereof. The host cell is then maintained under a condition which allows the polypeptide or composition of the invention to be produced.

In order to obtain transcription of the polynucleotide sequences herein within a host cell, a transcriptional regulatory region capable of driving gene expression in the target cell is utilized. The transcriptional regulatory region can comprise a promoter, enhancer, silencer or repressor element and is functionally associated with a nucleic acid of the present invention. Preferably, the transcriptional regulatory region drives high level gene expression in the target cell. Transcriptional regulatory regions suitable for use in the present invention include but are not limited to the human cytomegalovirus (CMV) immediate-early enhancer/promoter, the SV40 early enhancer/promoter, the JC polyomavirus promoter, the albumin promoter, PGK and the alpha.-actin promoter coupled to the CMV enhancer, the E. coli lac or trp promoters, the phage lambda P_{L} promoter and other promoters known to control expression of genes in prokaryotic or eukaryotic cells or their viruses. The expression vector can also contain a ribosome binding site for translation initiation and a transcription terminator.

In addition, the expression vectors may also contain a gene to provide a phenotypic trait for selection of transformed host cells such as dihydrofolate reductase or neomycin resistance for eukaryotic cell culture, or such as tetracycline or ampicillin resistance in E. coli.

In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example: (a) Bacterial: pQE70, pQE-9 (Qiagen), pBs, phagescript, PsiX174, pBluescript SK, pBsKS, pNH8a, pNH16a, pNH18a, pNH46a (Stratagene), pTrc99A, pKK223-3, pKK233-3, pDR540, and PRIT5 (Pharmacia); (b) Eukaryotic: pWLneo, pSV2cat, pOG44, pXT1, pSG (Stratagene) pSVK3, pBPV, PMSG, pSVL (Pharmacia) and pET20B. In one preferred embodiment, the vector is pET24B which is a kanamycin screening vector. However, any other plasmid or vector can be used as long as they are replicable and viable in the host.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, PL and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described construct. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology, 1986)).

The constructs in host cells can be used in a conventional manner to produce the polypeptide products encoded by the recombinant sequence. For example, the present invention contemplates methods for preparing a multi-unit complex that has angiostatic activity. Such method includes the steps of providing an expression vector encoding one or more tRNA synthetase fragments, transfecting a hot cell with such expression vector, and maintaining the host cell under condition suitable for expression. In preferred embodiments, an expression vector used to transfect a host cell encodes two or more tRNA synthetase fragments. More preferably, such tRNA synthetase fragments are tryptophanyl tRNA synthetase fragments. In some embodiments, such fragments are derived from mammalian tRNA synthetase, or more preferably, human tRNA synthetase. In some embodiments, the expression vector encodes a tRNA synthetase fragment selected from the group consisting of SEQ ID NOS: 12-17, 24-29, 36-41, 48-53, and any fragments, homologs, and analogs thereof. In some embodiments, such expression vector encodes a second tRNA synthetase fragment, wherein the second tRNA synthetase fragment is also selected from the group consisting of SEQ ID NOS: 12-17, 24-29, 36-41, 48-53, and any fragments, homologs, and analogs thereof. The two tRNA synthetase fragments can be different, homologous, substantially homologous, or identical.

The present invention also contemplates that a host cell (e.g., a bacteria) may or may not cleave the Methionine in the N-terminus of any of the polypeptides herein, depending upon the natural processes within the host cell. As such, it is further contemplated by the present invention that a composition can comprise of a combination of Met- and nonMet-tRNA synthetase fragments. For example, a bacteria transfected with a polynucleotide sequence encoding SEQ ID NO: 15 will result in a combination of both MetT2 and nonMetT2.

Alternatively, the polypeptides of the invention can be synthetically produced by conventional peptide synthesizers.

Proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook. et al., Molecular Cloning: A Laboratory Manual, Second Edition, Cold Spring Harbor, N.Y., (1989), the disclosure of which is hereby incorporated by reference.

Transcription of a polynucleotide sequence encoding the polypeptides of the present invention by higher eukaryotes is increased by inserting an enhancer sequence into the vector. Enhancers are cis-acting elements of DNA, usually about from 10 to about 300 base pairs (bp), that act on a promoter to increase its transcription. Examples include the SV40 enhancer on the late side of the replication origin (bp 100 to 270); a cytomegalovirus early promoter enhancer, a polyoma enhancer on the late side of the replication origin, and adenovirus enhancers.

Generally, recombinant expression vectors will include origins of replication and selectable markers permitting transformation of the host cell, e.g., the ampicillin resistance gene of E. coli, kanamycin for pET24B, and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Following transformation of a suitable host strain and growth of the host strain to an appropriate cell density, the selected promoter is derepressed by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites can be used to provide the required nontranscribed genetic elements.

Thus, in its most basic form, a polypeptide of the present invention can be prepared by providing the appropriate expression vector, transfecting a host cell with such expression vector, and maintaining the host cell under a condition suitable for expression. Preferably, expression vectors used herein include at least one nucleotide sequence encoding a tRNA synthetase fragment, or more preferably a tryptophanyl tRNA synthetase fragment, or any homolog or analog thereof. The vector encoding such tryptophanyl tRNA synthetase fragments may be modified to encode one or more non-naturally occurring cysteines in the dimerization domain of the polypeptide. In some embodiments, an expression vector encodes two or more tRNA synthetase fragments, or more preferably two or more tryptophanyl tRNA synthetase fragments. Such vectors preferably encode a linker situated between the first and second fragments.

Polypeptides are recovered and purified from recombinant cell cultures by methods used heretofore, including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. It is preferred to have low concentrations (approximately 0.1-5 mM) of calcium ion present during purification (Price, et al., J. Biol. Chem., 244:917 (1969)). Protein refolding steps can be used, as necessary, in completing configuration of the mature protein. Finally, high performance liquid chromatography (HPLC) can be employed for final purification steps. Additional purifications methods are disclosed herein.

b. Gene Therapy

The polynucleotides of the present invention can also be employed as gene therapy in accordance with the present invention by expression of such polypeptide in vivo.

Various viral vectors that can be utilized for gene therapy as taught herein include adenovirus, herpes virus, vaccinia, adeno-associated virus (AAV), or, preferably, an RNA virus such as a retrovirus. Preferably, the retroviral vector is a derivative of a murine or avian retrovirus, or is a lentiviral vector. The preferred retroviral vector is a lentiviral vector. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of additional retroviral vectors can incorporate multiple genes. All of these vectors can transfer or incorporate a gene for a selectable marker so that transduced cells can be identified and generated. By inserting a zinc finger derived-DNA binding polypeptide sequence of interest into the viral vector, along with another gene that encodes the ligand for a receptor on a specific target cell, for example, the vector is made target specific. Retroviral vectors can be made target specific by inserting, for example, a polynucleotide encoding a protein (dimer). Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the zinc finger-nucleotide binding protein polynucleotide.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsitation. Helper cell lines which have deletions of the packaging signal include but are not limited to .PSI.2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced. The vector virions produced by this method can then be used to infect a tissue cell line, such as NIH 3T3 cells, to produce large quantities of chimeric retroviral virions.

c. Zinc Fingers

Another targeted delivery system for polynucleotides encoding zinc finger derived-DNA binding polypeptides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, (1981)).

d. Targeted Liposomes

In some embodiments, targeted liposomes may be used to delivery the polynucleotides herein. In some embodiments, the polynucleotide sequence is an expression vector as described herein. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, (1988)).

The composition of the liposome is usually a combination of phospholipids, particularly high-phase-transition-temperature phospholipids, usually in combination with steroids, especially cholesterol. Other phospholipids or other lipids can also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes has been classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types. For example, a targeted liposome delivery system can include antibodies that specifically bind to cancer cells, tumor cells, photoreceptor cells, myocardial tissue, etc.

The surface of the targeted delivery system can be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

In general, the compounds bound to the surface of the targeted delivery system will be ligands and receptors which will allow the targeted delivery system to find and “home in” on the desired cells. A ligand can be any compound of interest which will bind to another compound, such as a receptor.

In general, surface membrane proteins which bind to specific effector molecules are referred to as receptors. In the present invention, antibodies are preferred receptors. Antibodies can be used to target liposomes to specific cell-surface ligands. For example, certain antigens expressed-specifically on tumor cells, referred to as tumor-associated antigens (TAAs), can be exploited for the purpose of targeting antibody-zinc finger-nucleotide binding protein-containing liposomes directly to the malignant tumor. Since the zinc finger-nucleotide binding protein gene product can be indiscriminate with respect to cell type in its action, a targeted delivery system offers a significant improvement over randomly injecting non-specific liposomes. A number of procedures can be used to covalently attach either polyclonal or monoclonal antibodies to a liposome bilayer. Antibody-targeted liposomes can include monoclonal or polyclonal antibodies or fragments thereof such as Fab, or F(ab′)₂, as long as they bind efficiently to an the antigenic epitope on the target cells. Liposomes can also be targeted to cells expressing receptors for hormones or other serum factors.

e. Cell Based Therapy

In any of the embodiments herein, cells transfected with the polynucleotides herein can be administered to a patient. In some embodiments, the cells transfected originate from the patient. In other embodiments, the cells transfected do not originate from the patient. In any event, the cells can be transfected by the constructs herein in vivo, ex vivo, or in vitro. In more preferred embodiments, the cells transfected are stem cells. Methods for making hematopoeituc stem cells are described in PCT/US2003/024839, which is incorporated herein by reference in its entirety.

III. Analogs

The present invention contemplates methods for screening for analogs for the compositions herein, and in particular, analogs for mini-TrpRS, T1, and T2. The term “analogs” as used herein means compounds that share structure and/or function, such as, for example, peptidomimetics, and any small or large organic or inorganic compounds. In preferred embodiments, an analog of the present invention is a small organic or inorganic compound that mimics the function and structure of mini-TrpRS, T1, or T2, by having similar interactions with their receptor(s).

1. Purification

In any of the embodiments herein, and especially for ophthalmic applications, the compositions (e.g., pharmaceutical formulation and/or polypeptides) herein are preferably substantially free of endotoxins.

The levels of endotoxins in a pharmaceutical or polypeptide preparation may be determined by any known technique; such techniques are widespread and commonly used by those of skill in the art in the pharmaceutical and biotechnology fields. For example, the FDA published Good Guidance Practices in February 1997 that noted several methods for quantifying endotoxin levels in a sample, including Limulus Amebocyte Lysate tests using chromogenic, endpoint-turbidimetric and kinetic-turbidimetric techniques. All of these techniques, as well as other techniques (including, but not limited to the use of rabbit pyrogen testing colonies) may be appropriately used to determine the endotoxin levels of the samples described herein.

Thus, in some embodiments, a pharmaceutical formulation or composition preferably has a concentration of endotoxins that is less than about 30, 25, 20, or 15, or more preferably less than about 10, 9, 8, 7, 6, 5, 4, 3, 2, or 1, or more preferably less than about 0.5, 0.1, 0.05, 0.01, 0.005, or 0.001 endotoxin units per milligram of polypeptide.

The amount of endotoxins in a sample refers to the amount of endotoxins (such as measured in endotoxin units or E.U.s) in a sample relative to the amount of desired polypeptide or pharmaceutical agent in that sample (generally provided per mg of polypeptide or pharmaceutical agent). The amount of endotoxins can be measured by any of a variety of techniques. However, the particular units employed herein are exemplary only, and are used throughout for reasons of consistency and readability. That is, the methods and materials presented herein are not limited by the particular “units” used to present the amount of endotoxins in a sample. Conversion between various units (by way of example only, E.U./mg of polypeptide to E.U./mL of sample) is considered well within the abilities of one of ordinary skill in the art.

In some embodiments, endotoxin reduction is the last or nearly last step in a purification process. In other embodiments, the endotoxin reduction step occurs at an early stage of the purification process (e.g., prior to steps that may lead to strong and/or irreversible binding of endotoxin to polypeptide).

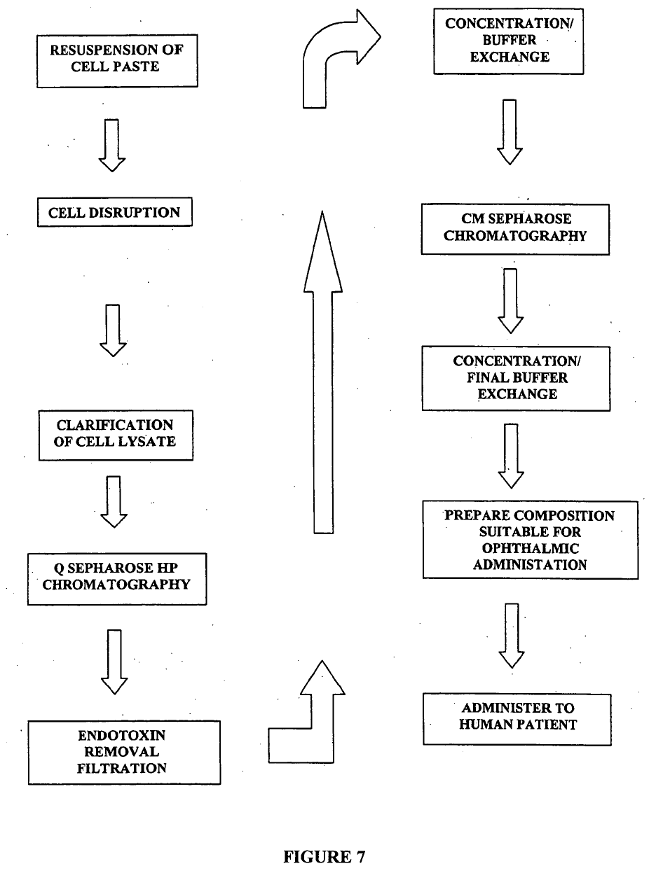

FIG. 7 illustrates a flowchart illustrating a sequence of purification steps (each occurring prior to the next) for purifying a pharmaceutical agent and/or polypeptide of the present invention. When a step occurs “prior to” another step, then the first step has been at least partially completed on a particular sample containing a polypeptide before the subsequent step is initiated.

At step 100 a cell paste is formed from cells grown in a fermentor (the cell paste may be properly stored until needed). Next at step 120, the cell paste is resuspended in a buffer. At step 130, the cells are disrupted.

At step 140, cell lysate is clarified. Clarification generally involves removal of insoluble matter (e.g., cellular debris, organelles and membranes) in all or in part from a solution containing a polypeptide of interest (e.g., a cell lysate or homogenate). The methods and compositions described herein are not limited by the technique used to produce the cell paste, lysate, or homogenate (or any other analogous term used in the art for the material). Clarification may be achieved by numerous methods known in the art, including by way of example only, simple filtration, centrifugation, dialysis, depth filtration, ultrafiltration using membranes with cut-offs in the vicinity of 100 K (in which the desired product is the filtrate and the retentate is discarded), decanting or other appropriate means known to those of skill in the art for such separations. That is, in general, after clarification, one fraction comprises mostly the insoluble portions of a cell, whereas the other fraction comprises mostly the soluble portions of a cell. In another aspect of a clarification step, a slurry becomes a clarified solution. It is of course appreciated by those in the art that endotoxin reduction does arise non-specifically during a clarification step by means of selecting against inclusion of remaining cell membranes (large fragments). However, clarification, by itself, will rarely, if ever, provide a polypeptide preparation that is substantially free of endotoxins.

In step 150, anion-chromatography is performed on the clarified cell lysate. This step can include collecting desired eluant fractions. Anion-exchange chromatography refers to the use of a positively charged surface with which a negatively-charged protein can form an ionic interaction. The protein may then be selectively eluted from the positively charged surface by manipulating the salt concentration and/or the pH of the eluting solvent. Examples of positively charged surfaces include anion-exchange resins.

Examples of anion exchange resins include, but are not limited to, diethylaminoethyl- (DEAE-), the quarternary ammonium- (Q- or QAE-), and the Amberlite-based resins. Different resin substrates, sizes (e.g., fast flow or FF, Source, or high performance or HP), and pore-diameters for anion-exchange resins are commercially available from standard chemical suppliers and their use is considered within the scope of the methods described herein. Preferably, an anion-exchange resin is selected from the group consisting of Q Sepharose, DEAE Sepharose, and ANX Sepharose. In still a further embodiment, the anion-exchange resin is Q-Sepharose. As is appreciated by those of skill in the art, smaller-sized resins may provide cleaner separation of products, but with a consequent trade-off in the speed with which such products are eluted from the chromatography column. Analyzing such trade-offs in selecting a anion-exchange resin is considered well within the ability of one of ordinary skill in the art. The anion-exhange chromatography is preferably performed prior to the reducing of the levels of endotoxins from the collected eluant.

Step 160 involves reducing the levels of endotoxins from the collected eluant fractions. Such step can remove all, substantially all, or some endotoxins from a sample. This step need not necessarily increase the overall purity of the protein (e.g., T1, T2, mini-trpRS). Techniques for endotoxin reduction include, by way of example, ultrafiltration (e.g., using membranes with cut-offs in the vicinity of 100 K in which the desired polypeptide product is in the retentate and the filtrate is discarded); reverse-phase, affinity, size-exclusion, hydrophobic interaction and/or anion-exchange chromatography (e.g., including Q Sepharose); sucrose centrifugation gradients; absorption of endotoxin onto activated charcoal, silica, hydroxyapatite, glass, and/or polystyrene; precipitation with isopropanol, ammonium acetate, or polyethylene glycol; phase-separation techniques using surfactants, such as detergents; use of charged-filter surfaces, and proprietary detoxifying media such as “Acticlean Etox™, Prosep-Remtox, Mustang E, and CUNO Zeta Plus ZA. The latter are typically provided in devices through which the polypeptide sample flows. In any of the embodiment herein, filtration-based techniques are preferable over column-based techniques.

In some embodiments, reducing the levels of endotoxins is made using ultrafiltration. Ultrafilteration involves separating all or at least some or at least one desired polypeptide(s) from different-sized molecules and/or molecules having a molecular weight different from the desired polypeptide(s). Ultrafiltration may involve a technique known as tangential flow filtration (as opposed to axial flow filtration). By passing the solution over the membrane in a tangential manner and having the ability to recirculate the solution (also called the retentate), the materials can pass through the membrane in a more gentle manner. The ability to pass through the membrane is determined by two factors: the membrane pore size (also known as the molecular weight cut-off), and the transmembrane pressure (set by the user by means of the pumps and valves). Using various embodiments of this set up, the protein of interest may either pass through the membrane (into the filtrate, this is used in clarification systems) or not pass through the membrane (stays in the retentate, this is used in buffer exchanges and concentration systems). In some embodiments, ultrafiltration is used to filter a liquid medium and small solute molecules through a semipermeable membrane having pores with an average cut-off molecular weight ranging from 100,000 to 1,000,000 Daltons, 200,000 to 900,000 Daltons, 300,000 to 800,000 Daltons, or 400,000 to 500,000 Daltons. In some embodiments, ultrafiltration is used to filter a liquid medium and small solute molecules through a semipermeable membrane having pores with an average cut-off molecular weight of at least 100,000 Daltons, at least 200,000 Daltons, at least 300,000 Daltons, at least 400,000 Daltons, at least 500,000 Daltons, at least 600,000 Daltons, at least 700,000 Daltons, at least 800,000, at least 900,000 or at least 1,000,000 Daltons. Performing an ultrafiltration step may include a dialysis process for separating globular proteins in solution from low-molecular weight solutes. Such a step can utilize a semipermeable membrane to retain protein molecules and allow small solute molecules and water to pass through. Dialysis membranes with molecular weight cut-offs ranging, by way of example only, from 100,000 to 1,000,000 Daltons, 200,000 to 900,000 Daltons, 300,000 to 800,000 Daltons, or 400,000 to 500,000 Daltons. In some embodiments, the molecular weight cut-offs may be at least 100,000 Daltons, at least 200,000 Daltons, at least 300,000 Daltons, at least 400,000 Daltons, at least 500,000 Daltons, at least 600,000 Daltons, at least 700,000 Daltons, at least 800,000 Daltons, at least 900,000, or at least 1,000,000 Daltons.

In preferred embodiments, the polypeptides purified by the present invention are not modified or denatured during the endotoxin-reduction process. The endotoxin-reduction step is preferably made prior to the buffer exchange step.

In step 170 the filtered eluant fractions are concentrated. Performing a concentration step can result in an increase of concentration of a desired polypeptide or pharmaceutical agent (e.g., any of the polypeptides herein) in the solvent by at least a factor of 2, 3, 4, 5, 6, 7, 8, 9, 10, 20, 30, 40, 50, 100, 200, 300, 400, or 500. Prepferably such a concentration-increasing process is conducted after a first chromatography step (e.g., anion-exchange and/or cation-exchange chromatography). Generally, a concentration step involves reducing the relative amount of solvent from a sample. Methods for affecting such a concentration step include, but are not limited to, ultrafiltration, evaporation, lyophilization, and precipitation (followed by resolubilization).

A concentration step will typically be performed at least once, 2, 3, 4, 5, or 6 times during the purification of a polypeptide and/or pharmaceutical agent. For example, if the first ion-exchange chromatography step is an anion-exchange chromatography step, then a concentration step may be performed on the amalgamation of eluted fractions containing the desired polypeptide and/or pharmaceutical agent (in this case, also known as the collected polypeptide fractions from the anion-exchange column). Similarly, if the second ion-exchange chromatography column is a cation-exchange chromatography step (also known as a polishing step), then a concentration step may be performed on the amalgamation of eluted fractions containing the desired polypeptide and/or pharmaceutical agent (in this case, the collected polished polypeptide fractions, or the collected polypeptide fractions from the cation-exchange column).

The concentration step(s) can occur either prior to a buffer exchange step or simultaneous to a buffer exchange step.

In step 180, buffer(s) are exchanged in preparation for a cation-exchange chromatography step. Buffer exchange involves chaning of a ‘solvent’ i.e., the liquid environment of a polypeptide is changed, in whole or in part. Solvents can include micromolecular solutes (e.g. salts) of the medium in which a desired polypeptide is found and/or macromolecule solutes. One suitable technique to perform a buffer exchange is ultrafiltration. Another suitable technique is dialysis of the solution containing the polypeptide against substantially larger quantities of a different buffer. Other buffer exchange techniques include, for example, gel permeation and diafiltration. The buffer exchange step can occur prior to, after, or simultaneously with a concentration step. One example of the latter approach is via the technique known as constant volume diafiltration. A buffer exchange step might be used once or multiple times in purifying a pharmaceutical agent and/or a polypeptide of the invention. By way of example only, if a particular polypeptide sample (i.e., T2) comprises an amalgamation of samples collected from an anion-exchange column (i.e., an anion-exchange chromatography step), then this polypeptide sample (known herein as a polypeptide sample in a post-anion exchange buffer) may undergo buffer exchange prior to loading the polypeptide sample through a cation-exchange column (i.e., a cation-exchange chromatography step). Another example wherein a buffer exchange step might be advantageously performed on a polypeptide sample is prior to storage of the finished polypeptide sample, but after the polishing step (e.g., the last ion-exchange chromatography step).

In step 190, a cation-exchange chromatography is performed. This step may include collection of desired eluant fractions. Cation-exchange chromatography refers to the use of a negatively charged surface with which the positively-charged protein can form an ionic interaction. When a cation-exchange chromatography step is performed on a sample that has already undergone an anion-exchange chromatography step, the cation-exchange chromatography step is sometimes referred to as a “polishing step”; the sample loaded onto the cation-exchange column is the unpolished sample and the eluted fractions containing the desired polypeptide sample have been polished and may be referred to as a polished polypeptide sample. The protein may then be selectively eluted from the negatively charged surface by manipulating the salt concentration and/or the pH of the eluting solvent. Examples of negatively charged surfaces include cation-exchange resins.

Examples of cation exchange resins include, by way of example only, carboxymethyl- (CM-) and sulfopropyl- (SP-) based resins. Different resin substrates, sizes (e.g., fast flow or FF, Source, or high performance or HP), and pore-diameters for cation-exchange resins are commercially available from standard chemical suppliers and their use is considered within the scope of the methods described herein. As is appreciated by those of skill in the art, smaller-sized resins may provide cleaner separation of products, but with a consequent trade-off in the speed with which such products are eluted from the chromatography column. Analyzing such trade-offs in selecting a cation-exchange resin is considered well within the ability of one of ordinary skill in the art.

Finally, at step 200, the sample is again concentrated and, oprionally, buffered are again exchanged. This results in a polypeptide sample that has reduced endotoxin levels. The low-endotoxin preparation may be further formulated in step 210 prior to administration to an organism (e.g., human) in step 220.

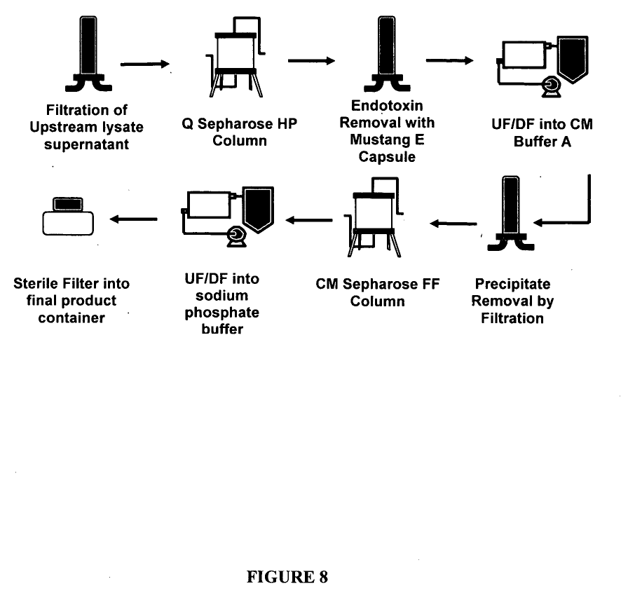

FIG. 8 is another illustration of the purification methods disclosed herein.

In some aspects of the methods herein, an endotoxin-reduction filtration step is performed after performing a clarification step and prior to performing a buffer exchange step. Furthermore, the endotoxin-reduction filtration step may be performed prior to performing a cation exchange chromatographic step. Alternatively, the endotoxin-reduction filtration step may be performed prior to performing a concentration step.

In some aspects of the methods herein, an endotoxin-reduction filtration step is performed after performing a clarification step and prior to performing a concentration step. Furthermore, the endotoxin-reduction filtration step may be performed prior to performing a cation exchange chromatographic step. Alternatively, the endotoxin-reduction filtration step may be performed prior to performing a buffer exchange step.

In some aspects of the methods herein, an endotoxin-reduction filtration step is performed after performing a clarification step and prior to performing a cation-exchange chromatographic step. Alternatively, the endotoxin-reduction filtration step may be performed prior to performing a concentration step. Alternatively, the endotoxin-reduction filtration step may be performed prior to performing a buffer exchange step.

In some aspects of the methods herein, an endotoxin-reduction filtration step is performed prior to performing a concentration step and prior to performing a cation-exchange chromatographic step and prior to a buffer exchange step.

The order of the concentration, buffer exchange, and cation-exchange chromatography steps in any of the purification methods herein may vary, but in one embodiment, at least one concentration step is performed prior to the buffer exchange step. Alternatively, a cation-exchange chromatographic step is performed after the buffer exchange step. Alternatively, at least one concentration step is performed prior to the cation-exchange chromatographic step. Alternatively, the cation-exchange chromatographic step is performed after a buffer exchange step and at least one concentration step. Alternatively, at least one concentration step is performed prior to the buffer exchange step and the cation-exchange chromatographic step. And alternatively, an additional concentration step is performed after any buffer exchange step.

In a further embodiment of any of the purification methods herein, the endotoxm-reduction filtration step is performed after an anion-exchange chromatographic step. In a further embodiment, the anion-exchange chromatographic step comprises use of an anion-exchange resin. In yet a further embodiment, the anion-exchange resin is selected from the group consisting of Q Sepharose, DEAE Sepharose, and ANX Sepharose. In still a further embodiment, the anion-exchange resin is Q Sepharose. In any of these uses of anion-exchange resins, a variety of grades and sizes may be used, including, but not limited to Source grade, fast flow grade and high performance grade.

In any of the purification methods herein, ae cation-exchange chromatographic step may comprise use of a cation-exchange resin. In a further embodiment, the cation-exchange resin is selected from the group consisting of CM Sepharose, SP Sepharose, and DEAE Sepharose. In still a further embodiment, the cation exchange resin is CM Sepharose. In any of these uses of cation-exchange resins, a variety of grades and sizes may be used, including, but not limited to Source grade, fast flow grade and high performance grade.

In an alternative aspect, methods for purifying a polypeptide can comprise an anion-exchange chromatographic step, a step comprising a means for reducing endotoxins, and a buffer exchange step, wherein the step comprising a means for reducing endotoxins is performed prior to the buffer exchange step. In a further embodiment, the polypeptide suitable for administration to a patient is suitable for ophthalmic administration. In still a further embodiment, the polypeptide suitable for ophthalmic administration is a modulator of angiogenesis. In yet a further embodiment, the polypeptide suitable for ophthalmic administration can be used to treat macular degeneration, diabetic retinopathy or diseases or conditions associated with unwanted ocular neovascularization. In a further refinement of any of the embodiments noted in this paragraph, the polypeptide is substantially free of endotoxins.

In some embodiments, purification of a polypeptide can comprise an anion-exchange chromatographic step, a step comprising a means for reducing endotoxins, and a buffer exchange step, wherein the step comprising a means for reducing endotoxins is performed prior to the buffer exchange step.

In any of the embodiments herein, a purification step can comprise of a concentration step of collected polished polypeptide fractions, wherein the collected polished polypeptide fractions are substantially free of endotoxins. In a further embodiment are methods of preparing the collected polished polypeptide fractions of the previous embodiment comprising performing a cation-exchange chromatographic step on an unpolished polypeptide sample thereby producing the collected polished polypeptide fractions of the previous embodiment, wherein the unpolished polypeptide sample is substantially free of endotoxins. In further embodiments are methods of producing the unpolished polypeptide sample of the previous embodiment comprising performing a buffer exchange step on a polypeptide sample in a post-anion exchange buffer thereby producing the unpolished polypeptide sample of the previous embodiment, wherein the polypeptide sample in the post-anion exchange buffer is substantially free of endotoxins. In further embodiments are methods of producing the polypeptide sample in the post-anion exchange buffer of the previous embodiment comprising performing a concentration step on collected polypeptide fractions from an anion-exchange column prior to the buffer exchange step thereby producing the polypeptide sample in the post-anion exchange buffer of the previous embodiment, wherein the collected polypeptide fractions from an anion-exchange column are substantially free of endotoxins. In further embodiments are methods of producing the collected polypeptide fractions from an anion-exchange column of the previous embodiment comprising performing an endotoxin-reduction filtration step prior to the concentration step of the previous embodiment. In a further embodiment are methods comprising performing an anion-exchange chromatographic step prior to the endotoxin-reduction filtration step.

The purity of the polypeptide sample may be ascertained before, during and/or after any of the aforementioned steps.

As described above a variety of host-expression vector systems may be utilized to express any of the polypeptide herein (e.g., a tRNA synthetase fragment, such as T1, T2, or miniTrpRS, preferably having a SEQ ID NO: 15-17, 27-29, 39-41, or 51-53). The expression systems that may be used for purposes include but are not limited to microorganisms such as bacteria (e.g., E. coli, B. subtilis) transformed with recombinant bacteriophage DNA, plasmid DNA or cosmid DNA expression vectors containing a polynucleotide sequence encoding any of the polypeptide herein at least in part; yeast (e.g., Saccharomyces, Pichia) transfected with recombinant yeast expression vectors containing a polynucleotide sequence encoding any of the polypeptide herein at least in part; insect cell systems infected with recombinant virus expression vectors (e.g., baculovirus) containing a polynucleotide sequence encoding any of the polypeptide herein at least in part; plant cell systems infected with recombinant virus expression vectors (e.g., cauliflower mosaic virus, CaMV; tobacco mosaic virus, TMV) or transfected with recombinant plasmid expression vectors (e.g., Ti plasmid) containing a nucleotide sequence encoding any of the polypeptide herein at least in part; or mammalian cell systems (e.g., COS, CHO, BHK, 293, 3T3, U937) harboring recombinant expression constructs containing promoters derived from the genome of mammalian cells (e.g., metallothionein promoter) or from mammalian viruses (e.g., the adenovirus late promoter; the vaccinia virus 7.5K promoter).

In eukaryotic systems, a number of selection systems may be used, including but not limited to genes such as the herpes simplex virus thymidine kinase (Wilkie et al., 1979, Nucleic Acids Res., 7:859-77), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) that can be employed in tk-, hprt- or aprt-cells, respectively. Also, antimetabolite resistance can be used as the basis of selection. The following genes exemplify this approach: dhfr, which confers resistance to methotrexate (Subramani S, et al., Mol Cell Biol. 1:854-64 (1981); Gasser et al., Proc Natl. Acad. Sci, 1982, 79(21):6522-26 (1982); O'Hare et al., (1981), Proc. Natl. Acad. Sci. USA 78:1527), especially in dhfr cells (Urlaub & Chasin, Proc. Natl. Acad. Sci, (1980), 77(7):4216-4220); gpt, which confers resistance to mycophenolic acid (Mulligan & Berg, (1981), Proc. Natl. Acad. Sci. USA 78:2072); neo, which confers resistance to the aminoglycoside G-418 (Colberre-Garapin et al., (1981), J. Mol. Biol. 150:1); hygro, which confers resistance to hygromycin (Santerre et al., (1984), Gene 30:147); the bar gene, which confers resistance to bialaphos; and D-amino acid oxidase, which confers resistance to D-alanine or D-serine (Erikson et al., Nat Biotechnol., (2004), 22(4):455-58).

In bacterial systems, a number of expression vectors may be selected depending upon the use intended for any of the polypeptide herein or homolog or analogs thereof. Suitable bacteria include, by way of example only, gram positive and gram-negative bacteria. In one embodiment, the polypeptide is expressed in Eschericia coli (“E. coli”) bacteria and subsequently isolated from the cells using the purification methods described herein.

The polypeptide can be expressed in a prokaryotic cell using expression systems known to those of skill in the art of biotechnology. Expression systems useful for the practice our methods and compositions are described in U.S. Pat. Nos. 5,795,745; 5,714,346; 5,637,495; 5,496,713; 5,334,531; 4,634,677; 4,604,359; 4,601,980, all of which are incorporated herein by reference in their entirety.

Prokaryotic cells can be grown under a variety of conditions known to the skilled artisan. In one aspect, the cells are grown in a medium suitable for growth of such cells, for example, minimal media or complete (i.e., rich) media. Generally, the medium used to grow the cells should not contain concentrations of salts or other chemicals, for example, urea, that are so high as to interfere with the partitioning of the polypeptide or with the formation of phases during the extraction methods.

Any of the polypeptide herein or homologs or analogs thereof may be expressed in transgenic animals. Animals species including, but not limited to, mice, rats, rabbits, guinea pigs, pigs, micro-pigs, goats, and non-human primates, e.g., baboons, monkeys, and chimpanzees may be used to generate transgenic animals expressing a transgene encoding any of the polypeptide herein or a homolog or analog thereof. Additionally, any of the polypeptide herein, including by way of example only, T1, T2, and miniTrpRS may be used in the compositions and methods described herein, may also be expressed in transgenic plants.

The purification methods herein are useful for purifying any of the polypeptide herein from a crude mixture that may be rich in contaminants, such as cell extracts or cellular debris. Cells that express the polypeptide herein can be prepared prior to the purification procedure in a variety of ways. For example, one may prepare a paste of frozen dead cells, or one may use living cells that are frozen, or living cells can be used directly in an extraction procedure.

If the polypeptide herein is purified from cells, the cells are disrupted or homogenized prior to extraction of the polypeptide. The purpose for disrupting or homogenizing the cells is to release the polypeptide herein from the cells. A variety of ways to disrupt or homogenize cells of diverse origin are well known in the art, for example, use of bead mills, osmotic shock, french presses, douncing, sonication, microfluidizing, high-pressure homogenization, and freeze fracture. If the polypeptide is secreted from the cells in which it is synthesized, the cells do not have to be lysed but the polypeptide can be extracted from the extracellular fluid or culture medium, e.g., a phase-forming agent may be added directly to the fermentor.

The purification methods described herein may include any techniques for separating the desired pharmaceutical agent or polypeptide from other undesired materials. These techniques include, by way of example only, tangential flow filtration (also known at TFF), depth filtration, ultrafiltration, dialysis, two-phase extractions, decantation, “salting out” techniques, an expanded bed adsorption system, and centrifugation.

In accordance with the compositions and purification methods described herein, the polypeptide can be purified from cells, a cell homogenate, disrupted cells, a crude mixture obtained following chemical synthesis of the polypeptide, or any kind of mixture that contains the polypeptide of interest and contaminants such that purification of the polypeptide is desirable.

Following each purification step, the polypeptide can be detected by a variety of methods including, but not limited to, bio assays, HPLC, amino acid determination or immunological assays, e.g., radioimmunoassay, ELISA, Western blot using antibody binding, SDS-PAGE. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, and epitope-binding fragments of any of the above.

The amount of the purified polypeptide and their level of purity can be determined by methods well known in the art. For example, and not by way of limitation, one may examine a polypeptide formulation that was prepared using our purification methods with polyacrylamide gel electrophoresis followed by staining the gel to visualize the total polypeptide in the gel. In one embodiment, the yield and purity of the polypeptide following two-phase extraction are determined using reverse phase HPLC.

The purity of a formulation of a polypeptide prepared using our purification methods may vary depending on the starting material. By way of example only, when purifying a polypeptide that is expressed in E. coli, the resulting preparation contains at least about 50% by weight of the polypeptide of interest, more preferably at least about 50%, more preferably at least about 70%, more preferably at least about 85% and preferably at least about 95%, preferaly at least about 96%, preferably at least about 97%, preferably at least about 98%%, preferably at least about 99%, or more preferably at least about 99.5%.

All polypeptide purification methods known to the skilled artisan may be used for further purification. Such techniques have been extensively described in Berger and Kimmel, Guide to Molecular Cloning Techniques, Methods in Enzymology, Volume 152, Academic Press, San Diego, Calif. (1987); Molecular Cloning: A Laboratory Manual, 2d ed., Sambrook, J., Fritsch, E. F., and Maniatis, T. (1989); Current Protocols in Molecular Biology, John Wiley & Sons, all Viols., (1989), and periodic updates thereof); New Polypeptide Techniques: Methods in Molecular Biology, Walker, J. M., ed., Humana Press, Clifton, N.J., (1988); and Polypeptide Purification: Principles and Practice, 3rd. Ed., Scopes, R. K., Springer-Verlag, New York, N.Y., (1987). Additional methods for further purifying the polypeptide include, but are not limited to ammonium sulfate precipitation, ion exchange, gel filtration, reverse-phase chromatography (and the HPLC or FPLC forms thereof), and hydrophobic interaction chromatography.

2. Library Screening

In one embodiment, a receptor of any of the compositions herein is used to screen for agents that can modulate the receptor. Preferably the agent is combined with a library of two or more candidate agents. Candidate agents that bind or interact with the receptor can be selected for further evaluation (e.g., by detecting ability to prevent/treat ocular neovascularization in mice or other mammals, see Examples 3 and 4). Examples of candidate agents include polypeptides (e.g., linear, cyclic, natural amino acids, unnatural amino acids, peptidomimetic compounds, and peptide nucleic acids), nucleic acids, carbohydrates, and small or large organic or inorganic molecules. Such libraries can be generated by a person of ordinary skill in the art and tailored for specific assays.

Candidate agents may be obtained from a wide variety of sources including libraries of synthetic or natural compounds. For example, numerous means are available for random and directed synthesis of a wide variety of organic compounds and bio-molecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, or amidification to produce structural analogs.

Agents that bind to the receptor can be then further evaluated for their angiostatic activity using any of the angiogenic assay models disclosed herein or otherwise known in the art. Examples of assays to determine angiogenesis include those described in Example 3 and the Metrigel angiogenesis assay described in Example 4. Agents which have a significant affect on angiogenesis are deemed analogs of the compositions herein.

3. Molecular Modeling

In some embodiments, the compositions may be modified or new compositions may be designed using computer modeling tools. Once there is confirmation of binding between a ligand (T2 or any of the other homodimers herein) and its receptor(s), modifications of the ligand may allow for increased binding capabilities or rational drug design.

This typically involves solving the crystal structure of the ligand/receptor complex; analyzing the contacts made between the ligand and receptor components; comparing how the ligand would interact with the receptor using computer stimulation and the appropriate software; and altering those portions of the ligand that are sterically hindered from or otherwise incompatible with binding to the ligand. The software typically utilized in molecular modeling is capable of achieving each of these steps, as well as suggesting potential replacements for various moieties of the ligand that would increase association with the native second kinase. Preferably, the software can also suggest small organic or inorganic compounds that can be used in lieu of the ligand (e.g., T2) to achieve the same affects.

In preferred embodiments, a molecular modeling system is used to analyze the interaction made by a tryptophanyl tRNA synthetase fragment and its receptor. Subsequently tryptophanyl tRNA synthetase fragment may be modified to improve the binding affinities of these two compounds.

One skilled in the art may use one of several methods to screen chemical moieties to replace portions of the ligand so that binding to the native second kinase is optimized. This process may begin by side-by-side visual inspection of the ligand and receptor on the computer screen based on the X-ray structure of the two compounds. Modified ligands may then be tested for their ability to dock to the native receptor using software such as DOCK and AUTODOCK followed by energy minimization and molecular dynamics with standard molecular mechanics force fields, such as CHARMM and AMBER.

Other specialized computer programs that may also assist in the process of replacement fragments include the following:

1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, Burlington, Mass.

3. AUTODOCK (D. S. Goodsell et al., “Automated Docking of Substrates to Proteins by Simulated Annealing”, Proteins: Structure, Function. and Genetics, 8, pp. 195-202 (1990)). AUTODOCK is available from Scripps Research Institute, La Jolla, Calif.

4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Other molecular modeling techniques may also be employed in accordance with this invention. See, e.g., N.C. Cohen et al., “Molecular Modeling Software and Methods for Medicinal Chemistry, J. Med. Chem., 33, pp. 883-894 (1990). See also, M. A. Navia et al., “The Use of Structural Information in Drug Design”, Current Opinions in Structural Biology, 2, pp. 202-210 (1992).

Once a compound has been designed or selected by the above methods, the efficiency with which that entity may bind to the receptor may be tested and further optimized by computational evaluation.

An entity designed or selected as binding to the native receptor may be further computationally optimized so that in its bound state it would preferably lack repulsive electrostatic interaction with the target receptor. Such non-complementary (e.g., electrostatic) interactions include repulsive charge-charge, dipole-dipole and charge-dipole interactions. Specifically, the sum of all electrostatic interactions between the ligand and the receptor when ligand is bound to the receptor preferably make a neutral or favorable contribution to the enthalpy of binding.

Specific computer software is available in the art to evaluate compound deformation energy and electrostatic interaction. Examples of programs designed for such uses include: Gaussian 92, revision C [M. J. Frisch, Gaussian, Inc., Pittsburgh, Pa. © 1992]; AMBER, version 4.0 [P. A. Kollman, University of California at San Francisco, © 1994]; QUANTA/CHARMM [Molecular Simulations, Inc., Burlington, Mass. © 1994]; and Insight II/Discover (Biosysm Technologies Inc., San Diego, Calif. © 1994). These programs may be implemented, for instance, using a Silicon Graphics workstation, Indigo₂ or IBM RISC/6000 workstation model 550. Other hardware systems and software packages will be known to those skilled in the art.

Once the modified ligand has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. Such substituted chemical compounds may then be analyzed for efficiency of fit to the receptor by the same computer methods described in detail, above.

IV. Pharmaceutical Formulations

Any of the compositions and analogs and any salts, prodrugs, or metabolites thereof, can be formulated for administration to an individual by the addition of a pharmaceutically acceptable carrier.

Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the drug.

Pharmaceutically acceptable salts include acid addition salts such as those containing sulfate, hydrochloride, phosphate, sulfonate, sulfamate, sulfate, acetate, citrate, lactate, tartrate, methanesulfonate, ethanesulfonate, benzenesulfonate, p-toluenesulfonate, cycloexylsulfonate, cyclohexylsulfamate, and quinate. Pharmaceutically acceptable salts can be obtained from acids such as hydrochloric acid, sulfuric acid, phosphoric acid, sulfonic acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Ophthalmically acceptable carriers are agents that have no persistent detrimental effect on the treated eye or the functioning thereof, or on the general health of the subject being treated. Typically, for intraocular methods of administration, a preservative will not be included in the formulation.

Useful aqueous suspensions for ophthalmic formulations can contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful ophthalmic formulations can also comprise of an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Ophthalmically acceptable solubilizing agent to aid in the solubility of any of the compositions herein include agents that result in the formation of a micellar solution or a true solution of the agent. Certain nonionic surfactants, for example polysorbate 80, can be useful as solubilizing agents, as can glycols, polyglycols, e.g., polyethylene glycol 400, and glycol ethers. In general, however, such surfactants and glycols are not used in compositions for intraocular administration except in very low doses because of their potential to cause certain harmful side effects, such as retinal detachment. Accordingly, such surfactants and glycols are preferably not used, or if required, in only small quantities.

Useful ophthalmically acceptable pH adjusting agents or buffering agents include, for example, acids such as acetic, boric, citric, lactic, phosphoric and hydrochloric acids;
bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

Useful ophthalmically acceptable salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Useful ophthalmically acceptable surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

The ophthalmic pharmaceutical formulations herein may also take the form of a solid article that can be inserted between the eye and eyelid or in the conjunctival sac, where it releases the agent. Release is to the lacrimal fluid that bathes the surface of the cornea, or directly to the cornea itself, with which the solid article is generally in intimate contact. Solid articles suitable for implantation in the eye in such fashion are generally composed primarily of polymers and can be biodegradable or non-biodegradable.

The pharmaceutical formulations herein can further include a therapeutic agent selected from the group consisting of: an antineoplastic agent, an anti-inflammatory agent, an antibacterial agent, an antiviral agent, an angiogenic agent, and an anti-angiogenic agent. Examples of such agents are disclosed herein.

For example, an antineoplastic agent may be selected from the group consisting of Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198 Hydroxyurea; Idarubicin Hydrochloride; Ifosfamide; Imofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycinl, Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

An anti-angiogenic agents are any agents that inhibit angiogenesis, whether disclosed herein or known in the art. In preferred embodiments, an anti-angiogenic agent is an anti-VEGF agent, such as Macugen™ (Eyetech, New York, N.Y.); or anti-VEGF antibody.

Pharmaceutical compositions can be formulated by standard techniques using one or more suitable carriers, excipients, and dilutents. See, e.g., Remington's Pharmaceutical Sciences, (19^{th} Ed. Williams & Wilkins, 1995) (incorporated herein by reference for all purposes).

Examples of suitable carriers, excipients and diluents include lactose, dextrose, sucrose, sorbitol, mannitol, starches, gum acacia, calcium phosphate, alginates, calcium silicate, microcrystalline cellulose, polyvinyl pyrrolidine, cellulose, tragacanth, gelatin syrup, methylcellulose, methyl and propyl hydroxybenzoates, talc, magnesium stearate, water and mineral oil. Other additives optionally include lubricating agents, wetting agents, emulsifying and suspending agents. An ophthalmic carrier is preferable in sterile, substantially isotonic aqueous solutions.

The pharmaceutical compositions may be formulated to provide immediate, sustained or delayed release of the compound. For applications providing slow release, certain carriers may be particularly preferred. Suitable slow release carriers may be formulated from dextrose, dextran, polylactic acid, and various cellulose derivatives, for example ethylhydroxycellulose in the form of microcapsules.

Various additives may be added to the formulations herein. Such additives include substances that serve for emulsification, preservation, wetting, improving consistency and so forth and which are conventionally employed in pharmaceutical preparations. Other additives include compounds that have surfactant properties, either ionic or non-ionic such as sorbitan monolaurate triethanolamine oleate, polyoxyethylenesorbitan monopalmitate, dioctyl sodium sulfosuccinate, monothioglycerol, thiosorbitol, ethylenediamine tetra-acetic acid, etc.

For non-ocular indications, an excipient may include a preservative. Suitable preservatives for use in non-ocular pharmaceutical preparations include benzalkonium chloride, benzethonium, phenylethyl alcohol, chlorobutanol, thimerosal and the like. Suitable buffers include boric acid, sodium and potassium bicarbonate, sodium and potassium borates, sodium and potassium carbonate, sodium acetate, sodium biphosphate, Tris, and the like, in amounts sufficient to maintain the pH between about pH 3 and about pH 9.5, most preferably between about pH 7 and pH 7.5. Suitable tonicity agents are dextran 40, dextran 70, dextrose, glycerin, potassium chloride, propylene glycol, sodium chloride and the like, such that the sodium chloride equivalent of the ophthalmic solution is in the range of 0.9.+−.0.2%.

Suitable antioxidant and stabilizers include sodium and potassium bisulfite, sodium and potassium metabisulfite, sodium thiosulfate, thiourea and the like. Suitable wetting and clarifying agents include polysorbate 80, polysorbate 20, poloxamer 282 and tyloxapol. Suitable viscosity increasing agents include dextran 40, gelatin, glycerin, hydroxyethyl cellulose, hydroxymethyl propyl cellulose, lanolin, methylcellulose, petrolatum, polyethylene glycol, polyvinyl alcohol, polyvinyl polyvinylpyrrolidone, carboxymethyl cellulose and the like. Stabilizers such as chelating agents that may be used include, for example, EDTA, EGTA, DTPA, DOTA, ethylene diamine, bipyridine, 1,10-phenanthrolene, crown ethers, aza crown, catechols, dimercaprol, D-penicillamine and deferoxamine. Antioxidants that may also act as stabilizers include such compounds as ascorbic acid, sodium bisulfite, ascorbyl palmitate, butylated hydroxyanisole, butylated hydroxytoluene, potassium metabisulfite and sodium metabisulfite.

Formulations can include capsules, gels, cachets, tablets, effervescent or non-effervescent powders or tablets, powders or granules; as a solution or suspension in aqueous or non-aqueous liquid; or as an oil-in-water liquid emulsion or a water-in-oil emulsion. Capsule or tablets can be easily formulated and can be made easy to swallow or chew. Tablets may contain suitable carriers, binders, lubricants, diluents, disintegrating agents, coloring agents, flavoring agents, flow-inducing agents, or melting agents. A tablet may be made by compression or molding, optionally with one or more additional ingredients. Compressed tables may be prepared by compressing the active ingredient in a free flowing form (e.g., powder, granules) optionally mixed with a binder (e.g., gelatin, hydroxypropylmethylcellulose), lubricant, inert diluent, preservative, disintegrant (e.g., sodium starch glycolate, cross-linked carboxymethyl cellulose) surface-active or dispersing agent. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, or the like. Tablets may optionally be coated or scored and may be formulated so as to provide slow- or controlled-release of the active ingredient. Tablets may also optionally be provided with an enteric coating to provide release in parts of the gut other than the stomach.

Formulations suitable for topical administration (e.g., wound healing) in the mouth wherein the active ingredient is dissolved or suspended in a suitable carrier include lozenges which may comprise the active ingredient in a flavored carrier, usually sucrose and acacia or tragacanth; gelatin, glycerin, or sucrose and acacia; and mouthwashes comprising the active ingredient in a suitable liquid carrier. Topical applications for administration according to the method of the present invention include ointments, cream, suspensions, lotions, powder, solutions, pastes, gels, spray, aerosol or oil. Alternately, a formulation may comprise a transdermal patch or dressing such as a bandage impregnated with an active ingredient and optionally one or more carriers or diluents.

To be administered in the form of a transdermal delivery system, the dosage administration will, of course, be continuous rather than intermittent throughout the dosage regimen. The topical formulations may desirably include a compound that enhances absorption or penetration of the active ingredient through the skin or other affected areas. Examples of such dermal penetration enhancers include dimethylsulfoxide and related analogs.

Formulations suitable for parenteral administration include aqueous and non-aqueous formulations isotonic with the blood of the intended recipient; and aqueous and non-aqueous sterile suspensions which may include suspending systems designed to target the compound to blood components or one or more organs. The formulations may be presented in unit-dose or multi-dose sealed containers, for example, ampoules or vials. For intraocular formulations, unit dosages are preferred because no preservatives are in the formulation. For other parenteral formulations, preservative may be used, which would allow for multi dose containers

Extemporaneous injections solutions and suspensions may be prepared from sterile powders, granules and tablets of the kind previously described. Parenteral and intravenous forms may also include minerals and other materials to make them compatible with the type of injection or delivery system chosen.

Particular parenteral administrations contemplated by the present invention include intraocular and intravitreous administrations to the eye. Pharmaceutical formulations for intraocular and intravitreous administrations include phosphate buffered saline (PBS) and balanced isotonic salt solution (BSS) with or without excipients such as mannitol or sorbitol as protein stabilizers.

In general, water, suitable oil, saline, aqueous dextrose (glucose), or related sugar solutions and glycols such as propylene glycol or polyethylene glycols are suitable carriers for parenteral solutions. Solutions for parenteral administration preferably contain a water soluble salt of the active ingredient, suitable stabilizing agents and, if necessary, buffer substances. Antioxidizing agents, such as sodium bisulfite, sodium sulfite, or ascorbic acid, either alone or combined, are suitable stabilizing agents. Also used are citric acid salts thereof, or sodium EDTA. In addition, parenteral solutions may contain preservatives, such as benzalkonium chloride, methyl- or propyl-paraben, or chlorobutanol. Suitable pharmaceutical carriers are described in Remington, cited supra.

In any of the embodiments herein, a composition or pharmaceutical formulation herein may by lypholized.

In any of the embodiments herein, the pharmaceutical formulations preferable have less than about 30, 20 or 10, more preferably less than 9, 8, 7, 6, 5, 4, 3, 2, or 1, or more preferably less 0.1, 0.01, or 0.001 endotoxin unit(s) per milligram of therapeutic agents

V. Indications

It is contemplated by the present invention that any of the compositions (including pharmaceutical formulations) herein may be used to modulate angiogenesis in a cell or tissue. Such methods involve contacting the cell or tissue with an appropriate anti-angiogenic (e.g., angiostatic) or angiogenic agent. For example, in some embodiments, a cell or tissue experiencing or susceptible to angiogenesis (e.g., an angiogenic condition) may be contacted with a multi-unit complex of a tRNA synthetase fragment, or a homolog or analog thereof to inhibit an angiogenic condition. In other embodiments, a cell or tissue experiencing or susceptible to insufficient angiogenesis (e.g., an angiostatic condition) may be contacted with an inhibitor of a tRNA synthetase fragment, e.g., an RNAi, antisense nucleic acid, antibody, or other binding agent or agent that interferes with angiostatic activity of a tryptophanyl-tRNA synthetase fragment.

The cells/tissue that may be modulated by the present invention are preferably mammalian cells, or more preferably human cells. Such cells can be of a healthy state or of a diseased state. In some embodiments, a cancerous cell, tumor cell, or a cell experiencing neovascularization is contacted with a composition of the present invention. In some embodiments, a cell experiencing angiogenesis due to an increase in VEGF, interferon gamma, and/or TNF-alpha, is contacted with a composition of the present invention. In one example, a photoreceptor cell is contacted with a multi-unit complex of the present invention.

Angiogenesis can be modulated in a cell or tissue by contacting the cell with a multi-unit complex, such as a dimer, trimer, etc. of the present invention. In preferred embodiments, such multi-unit complex is isolated. Furthermore, in any of the embodiments herein, a multi-unit complex may be soluble.

When modulating angiogenesis, the rate of angiogenesis may be inhibited by contacting a cell or tissue with an effective amount of a multi-unit complexes of the present invention. A of the multi-unit complexes of the present invention include a first monomer and a second monomer. The first and second monomers of the present invention may be different, homologous, substantially homologous, or identical to each other. Any of the monomers of the present invention can comprise a tRNA synthetase fragment. A tRNA synthetase fragment of the present invention can be, for example, a tryptophanyl tRNA synthetase fragment, a human tryptophanyl tRNA synthetase fragment, and/or any angiostatic fragment of a tRNA synthetase. Examples of angiostatic tryptophanyl tRNA synthetase fragments contemplated by the present invention include those selected from the group consisting of SEQ ID NOS: 12-17, 24-29, 36-41, 48-53, and any homologs and analogs thereof.

Units of a multi-unit complex may be covalently linked or non-covalently linked. Covalently linked monomers can be linked by any method disclosed herein, e.g., a linker, a disulfide bond. In some embodiments, two or more monomers are linked by one or more non-naturally occurring cysteines. Such cysteines are preferably located in a dimerization domain of a monomer. In some embodiments, monomers are linked by a linker. A linker of the present invention should be long enough to allow two or more monomers to freely rotate and dimerize with one another.

When modulating angiogenesis, the rate of angiogenesis may be enhanced by contacting a cell or tissue with an effective amount of an inhibitor of a tRNA synthetase fragment that has angiostatic activity. Examples of such inhibitors include, but are not limited to an antibody, an antisense nucleic acid, a RNAi nucleic acid, a peptidomimetic, a peptide nucleic acid, a peptide, and a small or large organic or inorganic molecule. Such inhibitors may function, for example, by competitively binding to a receptor of said tRNA synthetase fragment; binding to the binding site of said tRNA synthetase fragment; binding to said tRNA synthetase fragment and changing its conformation; inhibiting the expression of said tRNA synthetase, and/or inhibiting the cleavage of a full length tRNA synthetase which forms said tRNA synthetase fragment.

It is further contemplated by the present invention that any of the compositions herein may be administered to a patient susceptible to or suffering from a condition associated with increased angiogenesis (vascular formation) (“an angiogenic condition”) or a diminished capacity for vascular formation (“an anti-angiogenic condition”) (collectively, “angiogenesis-mediated conditions”).

Examples of angiogenic conditions that may be treated/prevented by the compositions/methods of the present invention include, but are not limited to, age-related macular degeneration (AMD), cancer (both solid and hematologic), developmental abnormalities (organogenesis), diabetic blindness, endometriosis, ocular neovascularization, psoriasis, rheumatoid arthritis (RA), and skin disclolorations (e.g., hemangioma, nevus flammeus, or nevus simplex).

Examples of anti-angiogenic conditions that may be treated/prevented by the compositions/methods of the present invention include, but are not limited to, cardiovascular disease (e.g., atherosclerosis (see Moulton, K., PNAS, Vol. 100, No. 8: 4736-4741 (2003)), restenosis (see Brasen J H., Arterioscler. Thromb. Vasc. Biol. November; 21(11):1720-6 (2001)), tissue damage after reperfusion of ischemic tissue or cardiac failure (see The U. of Tenn., The Vessel, 4(1) (2003)), chronic inflammation, and wound healing.

For example, the present invention relates to methods for treating or preventing conditions associated with ocular neovascularization using any of the compositions/methods herein. Conditions associated with ocular neovascularization include, but are not limited to, diabetic retinopathy, age related macular degeneration (“ARMD”), rubeotic glaucoma, interstitial keratitis, retinopathy of prematurity, ischemic retinopathy (e.g., sickle cell), pathological myopic, ocular histoplasmosis, pterygia, punitiate inner choroidopathy, and the like.

Examples of cancer that may be treatable or preventable by the compositions/methods herein include, but are not limited to, breast cancer; skin cancer; bone cancer; prostate cancer; liver cancer; lung cancer; brain cancer; cancer of the larynx; gallbladder; pancreas; rectum; parathyroid; thyroid; adrenal; neural tissue; head and neck; colon; stomach; bronchi; kidneys; basal cell carcinoma; squamous cell carcinoma of both ulcerating and papillary type; metastatic skin carcinoma; osteo sarcoma; Ewing's sarcoma; veticulum cell sarcoma; myeloma; giant cell tumor; small-cell lung tumor; gallstones; islet cell tumor; primary brain tumor; acute and chronic lymphocytic and granulocytic tumors; hairy-cell leukemia; adenoma; hyperpiasia; medullary carcinoma; pheochromocytoma; mucosal neuronms; intestinal ganglioneuromas; hyperplastic corneal nerve tumor; marfanoid habitus tumor; Wilm's tumor; seminoma; ovarian tumor; leiomyomater tumor; cervical dysplasia and in situ carcinoma; neuroblastoma; retinoblastoma; soft tissue sarcoma; malignant carcinoid; topical skin lesion; mycosis fungoide; rhabdomybsarcoma; Kaposi's sarcoma; osteogenic and other sarcoma; malignant hypercalcemia; renal cell tumor; polycythermia vera; adenocarcinoma; glioblastoma multiforme; leukemias (including acute myelogenous leukemia); lymphomas; malignant melanomas; epidermoid carcinomas; chronic myleoid lymphoma; gastrointestinal stromal tumors; and melanoma.

Methods of the present invention include a method for treating an individual suffering from an angiogenic condition by administering to the individual a pharmaceutical formulation comprising a multi-unit complex. A multi-unit complex of the present invention is a complex of 2 or more monomers, 3 or more monomers, 4 or more monomers, or 5 or more monomers.

In some embodiments, a monomer of a multi-unit complex is a tRNA synthetase fragment, or a horriolog or an analog thereof. Preferably, the tRNA synthetase fragment is a fragment of tryptophanyl tRNS synthetase (SEQ ID NO: 1), or any homologs or derivatives thereof. The tRNA synthetase fragment is preferably a fragment from a mammalian tRNA synthetase, or more preferably human tRNA synthetase. In some embodiments, a monomer of the multi-unit complex is selected from the group consisting of SEQ ID NOS: 12-17, 24-29, 36-41, and 48-53. A first monomer and a second monomer of the multi-unit complex can be different, homologous, substantially homologous, or identical. In preferred embodiments, a multi-unit complex is a dimer (with homologous or substantially homologous monomers), or more preferably a homodimer (with identical monomers).

The two or more monomers in a multi-unit complex may be covalently linked, non-covalently associated, or both.

It is further contemplated herein that the compositions herein can specifically interact with at least one angiogenic receptor. An angiogenic receptor is any cell surface receptor that can mediate angiogenesis (including abnormal developmental growth, tumorgenesis, lymphogenesis, and vasculogenesis). Angiogenic receptors of the present invention are preferably located on an endothelium cell, or more preferably vascular endothelium cell. In some embodiments, the compositions herein are used to modulate an angiogenic receptor or to treat an angiogenic-receptor mediated condition.

Known angiogenic receptors include, but are not limited to, growth factor receptors of VEGF, IGF, EGF, PDGF and FGF. Other preferred angiogenic receptors include cell adhesion molecules as described below. Angiogenic receptors also include CXC-receptors or chemokine receptors. Examples of CXC receptors include, but are not limited to, the group consisting of, IL8RA, IL8RB, IL8RBP, CXCR3, CXCR4, BLR1, and CXCR6. Examples of chemokine receptors include, but are not limited to, the group consisting of CCR1-CCR9, GPR2, CCRL1-CCRL2, and FPRL1.

In some embodiments, the methods of treatment disclosed herein further include administering to an individual suffering from an angiogenic condition one or more therapeutic agents selected from the group consisting of antineoplastic agents, antiviral agents, anti-inflammatory agents, antibacterial agents, anti-angiogenic agents, or anti-angiogenic agents.

Such combination treatments can be achieved by either administering to an individual a co-formulating of the compositions herein with the additional therapeutic agent(s) or by administering the compositions herein and the therapeutic agent(s) as two separate pharmaceutical formulations. In embodiments wherein more than one composition/therapeutic agent is administered to an individual, lower dosages of the compositions and/or therapeutic agent(s) may be utilized as a result of the synergistic effect of both active ingredients.

Examples of antineoplastic agents are provided herein and are known in the art.

Antibacterial agents that may be administered to an individual include, but are not limited to, penicillins, aminoglycosides, macrolides, monobactams, rifamycins, tetracyclines, chloramphenicol, clindamycin, lincomycin, imipenem, fusidic acid, novobiocin, fosfomycin, fusidate sodium, neomycin, polymyxin, capreomycin, colistimethate, colistin, gramicidin, minocycline, doxycycline, vanomycin, bacitracin, kanamycin, gentamycin, erythromicin and cephalosporins.

Anti-inflammatory agents that may be administered to an individual include, but are not limited to, NSAIDS (e.g., aspirin (salicylamide), sodium salicylamide, indoprofen, indomethacin, sodium indomethacin trihydrate, Bayer™, Bufferin™, Celebrex™, diclofenac, Ecotrin™, diflunisal, fenoprofen, naproxen, sulindac, Vioxx™), corticosteroids or corticotropin (ACTH), colchicine, and anecortave acetate.

Antiviral agents that may be administered to an individual include, but are not limited to, α-methyl-P-adamantane methylamine, 1,-D-ribofuranosyl-1,2,4-triazole-3 carboxamide, 9-[2-hydroxy-ethoxy]methylguanine, adamantanamine, 5-iodo-2′-deoxyuridine, trifluorothymidine, interferon, adenine arabinoside, CD4,3′-azido-3′-deoxythymidine (AZT), 9-(2-hydroxyethoxymethyl)-guanine (acyclovir), phosphonoformic acid, 1-adamantanamine, peptide T, and 2′,3′dideoxycytidine.

Angiogenic agents that may be administered to an individual include, but are not limited to, Angiogenin, Angiopoietin-1, Del-1, Fibroblast growth factors: acidic (aFGF) and basic (bFGF), Follistatin, Granulocyte colony-stimulating factor (G-CSF), Hepatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor-BB (PDGF-BB), Pleiotrophin (PTN), Progranulin, Proliferin, Transforming growth factor-alpha (TGF-alpha), Transforming growth factor-beta (TGF-beta), Tumor necrosis factor-alpha (TNF-alpha), and Vascular endothelial growth factor (VEGF)/vascular permeability factor (VPF).

Anti-angiogenic agents that may be administered to an individual include antagonists of angiogenic material. The term “antagonists of angiogenic material” is used herein to refer to any molecule that inhibiting the biological activity of an angiogenic material. Examples of antagonists of angiogenic material include, but are not limited to, antibodies that specifically bind the angiogenic material, iRNA that inhibit translation of the angiogenic material, and other agents that bind/interfere with the biological activity of the angiogenic material.

Examples of angiogenic materials include but are not limited to: (1) growth factors and their receptors; (2) remodeling and morphogenic receptors and their ligands; (3) adhesion receptors and their ligands; (4) matrix-degrading enzymes, such as Matrix-Metalo Proteinases (MMPs); (5) signaling molecules, such as Raf and MAPK, PKA, Rhos-family GTPases, PKB; and (6) transcription factors and regulators (e.g., hypoxia inducible factor (HIF)-1, Id ⅓, and Nuclear Factor-B) and homobox gene products (e.g., Hox D3, and B3).

In some embodiments, the angiogenic material is a growth factor and/or its receptor. Examples of growth factors receptors include VEGF receptors (e.g., soluble VEGFR1, VEGFR1 (Flt-1), VEGFR2 (Flk-1), and VEGFR3 (Flt-4)) and their ligands (e.g., VEGF A, B, C, and D). Thus, in some embodiments, an anti-angiogenic agent is an antagonist to a VEGF receptor, such as VEGFR1, VEGFR2, VEGFR3, or an antagonist to a VEGF ligand, such as VEGFA, VEGFB, VEGFC, or VEGFD. In some embodiments, an anti-angiogenic agent is antagonist to a VEGF ligand (e.g., VEGFA-VEGFD). More preferably, an anti-angiogenic agent is antagonist to VEGFA. Examples of anti-VEGF, anti-angiogenic agents include Avastin (Genentech, Inc.), Macugen (EyeTech Pharmaceuticals, Inc.) or Visudyne (Novartis, Crop.) and anti-VEGF monoclonal antibody M293. Additional examples of anti-VEGF anti-angiogenic agents are disclosed in U.S. Pat. Nos. 5,730,977, 6,383,484, 6,403,088, 6,479,654, 6,559,126, and 6,676,941, all of which are incorporated herein by reference for all intended purposes.

Additional examples of growth factors and their receptors include, but are not limited to, angiogenin, angiopoietin-1, Del-1, fibroblast growth factors (“FGF”) and FGFR (including acidic aFGF and basic bFGF), follistatin, granulocyte colony-stimulating factor (G-CSF), hepatocyte growth factor (HGF), Interleukin-8 (IL-8), leptin, midkine, placental growth factor, platelet-derived endothelial growth factor (PD-ECGF), platelet-derived growth factor-BB (PDFG-BB), pleiotrophin (PTN), progranulin, proliferin, transforming growth factor (TGF)-alpha, TGF-beta, and tumor necrosis factor (TNF)-alpha.

In some embodiments, an anti-angiogenic agent of the present invention is an antagonist of a remodeling and morphogenic receptor and/or ligand. Examples of remodeling and morphogenic receptors and ligands include, but are not limited to, the Tie receptors (e.g., Tiel and Tie2) and their ligands (e.g., ANG-1, ANG-2, and ANG-¾), as well as the Ephrin receptors (e.g., EphB1, EphB2, EphB3, EphB4, EphB6, EphA4) and their ligands (e.g., ephrin B1, B2, and B3).

In some embodiments, an anti-angiogenic agent of the present invention is an antagonist of an adhesion receptor and/or its ligand. Examples of adhesion receptors and their ligands include, but are not limited to, the integrins, cadherins, semophorins, and fibronectin. There are eighteen alpha and eight beta mammalian subunits which assemble to form 24 different heterodimers of integrin receptors. In some embodiments, an antagonist of an adhesion receptor is an antagonist of a vascular integrin receptor selected from the group consisting of α1β1, α2β1, α3 β1, α4β1, α5β1, α6β1, α8β1, α9β1, αVβ1, αVβ3, αVβ5, α6β4, and αVβ8. In more preferred embodiments, an antagonist of an adhesion receptor is an antagonist of a vascular integrin receptor selected from the group consisting of α1β1, α2β1, α5β1, and αVβ3. In more preferred embodiments, an antagonist of an adhesion receptor is an antagonist of αVβ3.

Peptide and antibody antagonists of this integrin inhibit angiogenesis by selectively inducing apoptosis of the proliferating vascular endothelial cells. Integrin antibodies are commercially available from, e.g., Chemicon Internation, Biocompare, Soretec, etc.

Two cytokine-dependent pathways of angiogenesis exist and can be defined by their dependency on distinct vascular cell integrins, αVβ3 and αVβ5. Specifically, basic FGF- and VEGF-induced angiogenesis depend on integrin αVβ3 and αVβ5, respectively, since antibody antagonists of each integrin selectively block one of these angiogenic pathways in the rabbit corneal and chick chorioallantoic membrane (CAM) models. Peptide antagonists that block all aV integrins inhibit FGF- and VEGF-stimulated angiogenesis. While normal human ocular blood vessels do not display either integrin, αVβ3 and αVβ5 integrins are selectively displayed on blood vessels in tissues from patients with active neovascular eye disease. While only αVβ3 was consistently observed in tissue from patients with ARMD, αVβ3 and αVβ5 both were present in tissues from patients with PDR. Systemically administered peptide antagonists of integrins blocked new blood vessel formation in a mouse model of retinal vasculogenesis.

There are many different types of cadherins. The most extensively studied group of cadherins is known as the classical, or type I, cadherins. Cadherins that contain calcium binding motifs within extracellular domain cadherin repeats, but do not contain an HAV CAR sequence, are considered to be nonclassical cadherins. To date, nine groups of nonclassical cadherins have been identified (types II-X). These cadherins are membrane glycoproteins. Type II, or atypical, cadherins include OB-cadherin, also known as cadherin-11 (Getsios et al., Developmental Dynamics 211:238-247, (1998)); cadherin-5, also known as VE-cadherin (Navarro et al., J. Cell Biology 140:1475-1484 (1998)); cadherin-6, also known as K-cadherin (Shimoyama et al., Cancer Research 55:2206-2211 (1995)); cadherin-7 (Nakagawa et al., Development 121:1321-1332 (1995); cadherin-8 (Suzuki et al., Cell Regulation 2:261-270 (1991)), cadherin-12, also known as Br-cadherin (Tanihara et al., Cell Adhesion and Communication 2:15-26, (1994)); cadherin-14 (Shibata et al., J Biological Chemistry 272:5236-5240 (1997)), cadherin-15, also known as M-cadherin (Shimoyama et al., J. Biological Chemistry 273:10011-10018 (1998)), and PB-cadherin (Sugimoto et al., J. Biological Chemistry 271:11548-11556 (1996)). For a general review of atypical cadherins, see Redies and Takeichi, Developmental Biology 180:413-423 (1996) and Suzuki et al., Cell Regulation 2:261-270 (1991).

Additional examples of angiogenic receptors include neuropillins (e.g., neuropillin-1 and neuropillin-2), endoglin, PDFGβR, CXCR-4, Tissue Factor (“TF”), thrombin receptor, Gα₁₃, and EP3. It has been suggested that T-2 also binds to neuropillin-1 and 2, see, e.g., International Appl. No. PCT/US02/23868, having publication No. WO 03/009813, which is incorporated herein by reference. Thus, the present invention contemplates methods for identifying other binding partners that can specifically interact with and/or bind tRS, or more preferably T2. Such methods include the use of a yeast two hybrid system, a phage display library system, screening peptide libraries, computer imaging programs, and the like.

In any of the embodiments herein, anti-angiogenic agents can include nucleic acids, polypeptides, peptidomimetics, PNAs, antibodies, fragments of antibodies, small or large organic or inorganic nucleic acids that bind to angiogenesis associated molecules.

Other known anti-angiogenic agents that are found in the body include, but are not limited to, angioarrestin, angiostatin (plasminogen fragment), antiangiogenic antithrombin III, cartilage-derived inhibitor (CDI), CD59 complement fragment, endostatin (collagen XVIII fragment), fibronectin fragment, Gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kDa fragment, proligerin-related protein (PRP), retinoids, tetrahydrocortisol-S, thrombosponrin-1 (TSP-1), transforming growth factor-beta, vasculostatin, vasostatiri (calreticulin fragment).

VI. Administration

Administration of a composition of the present invention to a target cell in vivo can be accomplished using any of a variety of techniques well known to those skilled in the art.

For example, compositions of the present invention can be administered systemically or locally by any means known in the art (e.g., orally, intraocularly, intravascularly (i.v.), intradermally, intramuscularly, transdermally, transmucosally, enterically, parentally, by inhalation spray, rectally, or topically) in dosage unit formulations and containing conventional pharmaceutically acceptable carriers, adjuvants, and vehicles.

For purposes of this invention the term “ophthalmic administration” encompasses, but is not limited to, intraocular injection, subretinal injection, intravitreal injection, periocular administration, subconjuctival injections, retrobulbar injections, intracameral injections (including into the anterior or vitreous chamber), sub-Tenon's injections or implants, ophthalmic solutions, ophthalmic suspensions, ophthalmic ointments, ocular implants and ocular inserts, intraocular solutions, use of iontophoresis, incorporation in surgical irrigating solutions, and packs (by way of example only, a saturated cotton pledget inserted in the formix).

As used herein the term parenteral as used herein includes, subcutaneous, intravenous, intramuscular, intrastemal, infusion techniques or intraperitoneally. Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

The dosage regimen for treating a disorder or a disease with the vectors of this invention and/or compositions of this invention is based on a variety of factors, including the type of disease, the age, weight, sex, medical condition of the patient, the severity of the condition, the route of administration, and the particular compound employed. Thus, the dosage regimen can vary widely, but can be determined routinely using standard methods.

For systemic administration, the polypeptides (preferably dimers or homodimers) and/or small molecules of the present invention are preferably administered at a dose of at least 0.05, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 20, 30, 40, 50, 75, 100, or 150 mg/kg body weight. In other embodiments, the polypeptides (preferably dimers or homodimers) and/or small molecules herein are administered systemically at a dose of 0.1-100 mg/kg, more preferably 0.5-50 mg/kg, more preferably 1-30 mg/kg body weight, or more preferably 5-20 mg/kg.

For localized administration, the polypeptides (preferably dimers or homodimers) and/or small molecules of the present invention are preferably administered at a dose of at least 50 μg, 100 μg, 150 μg, 200 μg, 250 μg, 300 μg, 350 μg, 400 μg, 450 μg, 500 μg, 550 μg, 600 μg, 650 μg, or 700 μg. In other embodiments, the polypeptides (preferably dimers or homodimers) and/or small molecules herein are administered locally at a dose of 50-1000 μg, more preferably 100-800 μg, more preferably 200-500 μg, or more preferably 300-400 μg per site.

For example, for dermal administration the polypeptides (e.g., dimers) and/or peptidomimetics and/or small molecules of the present invention are administered at a dose of 50-1000 μg/cm², more preferably 100-800 μg/cm², or more preferably 200-500 μg/cm². In another example, for ocular administration, the polypeptides (e.g., dimers) and/or peptidomimetics and/or small molecules of the present invention are administered at a dose of 50-1000 μg/eye, more preferably 100-800 μg/eye, or more preferably 200-500 μg/eye.

The pharmaceutical compositions preferably include the active ingredient (e.g., T2) in an effective amount, i.e., in an amount effective to achieve therapeutic or prophylactic benefit. The actual amount effective for a particular application will depend on the condition being treated and the route of administration. Determination of an effective amount is well within the capabilities of those skilled in the art, especially in light of the disclosure herein.

Preferably, the effective amount of the active ingredient, e.g., T2, is from about 0.0001 mg to about 500 mg active agent per kilogram body weight of a patient, more preferably from about 0.001 to about 250 mg active agent per kilogram body weight of the patient, still more preferably from about 0.01 mg to about 100 mg active agent per kilogram body weight of the patient, yet still more preferably from about 0.5 mg to about 50 mg active agent per kilogram body weight of the patient, and most preferably from about 1 mg to about 15 mg active agent per kilogram body weight of the patient.

In terms of weight percentage, the formulations of the present invention will preferably comprise the active agent, e.g., T2, in an amount of from about 0.0001 to about 10 wt. %, more preferably from about 0.001 to about 1 wt. %, more preferably from about 0.05 to about 1 wt. %, or more preferably about 0.1 wt. to about 0.5 wt. %.

VII. Screening/Diagnosis

In any of the embodiments herein a cell or tissue may be screened for an angiogenesis mediated condition (e.g., an anti-angiogenic condition or an angiogenic condition). This can be accomplished by any technology known in the art. For example, tagged probes, tagged probes described in WO 2004/011900, which is incorporated herein by reference for all purposes, may be used to identify and/or quantify angiostatic and/or angiogenic tRNA synthetase fragments in a sample. Generally, such tagged probes include a binding moiety that is specific to a tRNA synthetase fragment (e.g., miniTrp-RS, T1, or T2), a detectable reporter (such as a fluorescent group), and optionally a mobility modifier. The mobility modifier and detectable reporter are linked to the binding moiety by a cleavable linker. The binding moiety can be, for example, an antibody specific to a tRNA synthetase fragment disclosed herein (e.g., a polypeptide selected from SEQ ID NOS: 12-17, 24-29, 36-41, 48-53, and any homologs and analogs thereof.

After binding the target agent, the cleavable tags can be cleaved and separated according to their mobility. More than one tagged probe may be used simultaneously to determine the angiogenic state of a cell/tissue/organism.

In some embodiments, patient may be diagnosed or screened for one or more conditions associated with angiogenesis (an angiogenesis mediated condition) prior to or subsequent a treatment. For example, an individual may be screened for a condition selected from the group consisting of adiposity, cardiovascular diseases, restenosis, cancer, chronic inflammation, tissue damage after reperfusion, neurodegeneration, rheumatoid arthritis, Crohn's disease, Alzheimer's disease, Parkinson's disease, diabetes, endometriosis, psoriasis, failure in wound healing, and ocular neovascularization. If a patient is diagnosed as having such a condition or being susceptible to such a condition, a therapeutically effective amount of the compositions herein may be administered to the patient. Similarly, a patient may be monitored after a therapeutic treatment is administered to see if additional treatments are required.

Methods for diagnosing or screening patients for conditions are known in the art and include detection of single nucleotide polymorphisms (SNPs) or alleles that are associated with resistance or susceptibility to such conditions. In preferred embodiments, such diagnosis is made using a microarray device. Examples of SNPs that may be used to detect/diagnose an individual with an ocular neovascular condition (or susceptibility thereof) are disclosed in U.S. Pat. No. 6,713,300, which is incorporated herein by reference. Additional SNPs related to angiogenesis-mediated conditions can be identified on the dbSNP database maintained by NCBI at <http://www.ncbi.nlm.nih.gov>.

VIII. Business Methods

The invention herein also contemplates business methods by providing therapeutics and/or diagnostics for treating individuals suffering from or susceptible to angiogenic conditions. In some embodiments, a business method of the present invention contemplates searching for an agent that modulates or binds to a receptor of tRNA synthetase fragment and commercializing such an agent. A tRNA synthetase fragment is preferably a tryptophanyl tRNA synthetase fragment. The tryptophanyl tRNA synthetase fragments herein are preferably mammalian, or more preferably human. Examples of human tryptophanyl tRNA synthetase fragments include but are not limited to SEQ ID NOS: 12-17, 24-29, 36-41, and 48-53. Preferably a tRNA synthetase fragment herein is angiostatic. In some embodiments, the step of searching for an agent that modulates or binds to a receptor of tRNA synthetase fragment involves using a computer program to generate peptidomimetics of the tRNA synthetase fragment. In some embodiments the step of searching involves screening a library of candidate agents to identify an agent that modulates or binds to the receptor. There are various forms of libraries available for screening candidate agents. Such libraries include peptide libraries, and small molecule libraries, as well as others disclosed herein or known in the art.

The present invention also contemplates a business method that includes the steps of modifying a tRNA synthetase fragment to enhance its dimerization capabilities and commercializing the enhanced fragment or dimer form thereof. Again, the tRNA synthetase fragment can be tryptophanyl tRNA synthetase fragment, or more preferably a fragment selected from the group consisting of NOS: 12-17, 24-29, 36-41, and 48-53. In some embodiments, such business methods contemplate the use of a computer program to optimize the tRNA synthetase fragments herein. Examples of computer programs that can be used to optimize a ligand include, but are not limited to GRID, MCSS, AUTODOCK, DOCK, AMBER, QUANTA, and INSIGHT II. In other embodiments, the business methods herein contemplate generating an expression vector that encodes a tRNA synthetase fragment modified to include one or more non-naturally occurring cysteines. Preferably, such modifications occur in the dimerization domain of the fragment. In other embodiments, the business methods herein contemplate generating an expression vector that encodes two tRNA synthetase fragments. Such vectors can also encode a linker that is preferably situated between the two fragments.

The business methods herein also contemplate commercializing fragments of a tRNA synthetase that modulate angiogenesis. In some embodiments, such fragments may inhibit angiogenesis (e.g., angiostatic fragments of a tRNA synthetase). In other embodiments, such fragments may enhance angiogenesis (.e.g., inhibitors of angiostatic fragments of a tRNA synthetase).

In one embodiment, the present invention relates to a business method which includes the steps of expressing an expression vector encoding a tRNA synthetase fragment and commercializing said fragment for modulating angiogenesis. A tRNA synthetase fragment of the present invention can be, for example, a tryptophanyl tRNA synthetase fragment, a human tRNA synthetase fragment, or any angiostatic fragment of a tRNA synthetase. Examples of such fragments include but are not limited to SEQ ID NOS: 12-17, 24-29, 36-41, 48-53, and any homologs and analogs thereof.

In some embodiments, the fragments commercialized are part of a multi-unit complex. A multi-unit complex of the present invention can include two or more monomer units covalently bound or non-covalently associated.

In some embodiments, the expression vector also encodes a second tRNA synthetase fragment. The first tRNA synthetase fragment and the second tRNA synthetase fragment can be different, homologous, substantially homologous, or identical. Moreover, in some embodiments, the first tRNA synthetase fragment and the second tRNA synthetase fragment are modified to include at least one non-naturally occurring cysteine. Such non-naturally occurring cysteine is preferably situated in the dimerization domain of the tRNA synthetase fragments.

An expression vector encoding two or more tRNA synthetase fragments can have the two or more fragments aligned in tandem. In some embodiments, the expression vector can also encode a linker. The polynucleotide sequence encoding the linker can be situated between the sequence encoding the first and the sequence encoding the second tRNA synthetase fragments. A linker of the present invention is preferably sufficiently long to allow said first and said second tRNA synthetase fragments to free rotate and dimerize.

The fragments and multi-unit complexes herein can be prepared by tranfecting a host cell with the expression vectors disclosed herein, and maintaining the host cell under a condition that permits the expression of the one or more tRNA synthetase fragments.

The business methods herein also contemplate commercializing diagnostics for detection of angiogenesis-mediated conditions (e.g., either an angiostatic or angiogenic condition).

For example, a diagnostic may be commercialized to detect an angiogenic condition, such as an ocular neovascularization condition or AMD, either independently or in combination with an angiostatic composition disclosed herein (e.g., an angiostatic fragment of a tRNA synthetase, more preferably an angiostatic fragment of a tryptophanyl tRNA synthetase, or more preferably mini-trpRS, T1 and/or T2). Examples of genetic variations and diagnostics that may be used to detect ocular neovascularization conditions include those disclosed in U.S. Pat. No. 6,713,300, which are incorporated herein by reference for all purposes.

In another example, a diagnostic may be commercialized to detect an anti-angiogenic condition, such as a cardiovascular disease, either independently or in combination with an angiogenic composition disclosed herein (e.g., an inhibitor of an angiostatic fragment of a tRNA synthetase, such as a tryptophanyl tRNA synthetase, e.g., mini-trpRS, T1 and/or T2).

In any of the embodiments herein further contemplate the step of partnering with a third party partner to commercialize the compositions and/or diagnostics herein. Examples of partners can include biotech partners, pharmaceutical partners, consumer products partners, agricultural partners, scientific partners, government partners, etc.

In some embodiments, partners can provide funding or research capabilities to, for example, discover analogs of the compositions herein, discover receptors for the compositions herein, optimize the compositions, run clinical trials on the compositions herein, develop inhibitors for the compositions herein, etc.

IX. Kits

The invention also provides a kit comprising one or more containers filled with one or more of the compositions herein. The kits can include written instructions on how to use such compositions (e.g., to modulate angiogenesis or treat a patient suffering from an angiogenic condition).

In one embodiment, a kit comprises a container wherein the container comprises one or more of the compositions herein. Examples of compositions that may be in a container include: a composition comprising an isolated tRNA synthetase fragment comprising, consisting essentially of, or consisting of an amino acid sequence selected from the group consisting of SEQ ID NOS: 12-17, 24-29, 36-41, 48-53 and any homologs or analogs thereof. If a tRNA synthetase fragment comprises of, consists essentially of, or consists of SEQ ID NOS: 12, 15, 24, 27, 36, 39, 48, 51 or any homologs or analogs thereof, then such tRNA synthetase fragment is preferable less than 45 kD, more preferably less than 44 kD, 43.9 kD, 43.8 kD, 43.7 kD, 43.6 kD, or more preferably less than 43.5 kD. If a tRNA synthetase fragment comprises of, consists essentially of, or consists of SEQ ID NOS: 13, 16, 25, 28, 37, 40, 49, 52, or any homologs and analogs thereof, then such tRNA synthetase fragment is preferably less than 48 kD, more preferably less than 47 kD, or more preferably less than 46 kD. If a tRNA synthetase fragment comprises of, consists essentially of, or consists of SEQ ID NOS: SEQ ID NO: 14, 17, 26, 29, 38, 41, 50, 53, or any homologs or analogs thereof, then such tRNA synthetase fragment is preferably less than 53 kD, more preferably less than 52 kD, more preferably less than 51 kD, more preferably less than 50 kD, or more preferably less than 49 kD. Preferably a tRNA synthetase fragment in a container is purified.

In some embodiments, a kit of the present invention comprises a container comprising a multi-unit complex, wherein at least one unit of the multi-unit complex comprises a tRNA synthetase fragment or a homolog or analog thereof. A multi-unit complex can be, for example, a dimer having two units. Monomers of a multi-unit complex can be different from each other, homologous, substantially homologous, or identical. In some embodiments, a multi-unit complex is a dimer having two homologous monomers.

In any of the embodiments herein a tRNA-synthetase fragment can be a tryptophanyl tRNA synthetase fragment, a human tryptophanyl tRNA-synthetase, and/or any angiostatic fragment of a tRNA synthetase fragment. For example, a tRNA synthetase fragment can be selected from the group consisting of SEQ ID NOS: 12-17, 24-29, 36-41,48-53, and any homologs or analogs thereof.

Any two monomers within a multi-unit complex may be covalently linked or non-covalently linked. The composition in the first container may be packaged for systemic administration in a single unit dosage. When packaged in single unit dosages, a dose may range between 50-1000 μg/dose.

The kit herein may also include a second therapeutic agent. Such second therapeutic agent may be contained in a second container. Examples of a second therapeutic agent include, but are not limited to an antineoplastic agent, an anti-inflammatory agent, an antibacterial agent, an antiviral agent, an angiogenic agent, and an anti-angiogenic agent. In preferred embodiments, a second therapeutic agent is an anti-angiogeneic agent.

In some embodiments, a kit of the present invention comprises a container comprising a composition of a first tRNA synthetase fragment and a second tRNA synthetase fragment wherein the first tRNA synthetase fragment has a methionine at its N-terminus and wherein the second tRNA synthetase fragment does not have a methionine at its N-terminus; and written instructions for use thereof.

The first tRNA synthetase fragment can be, for example, a tryptophanyl tRNA synthetase fragment, a human tRNA synthetase fragment, or an angiostatic fragment of a tRNA synthetase. The second TRNA synthetase fragment can be, for example, a tryptophanyl tRNA synthetase fragment, a human tRNA synthetase fragment, or an angiostatic fragment of a tRNA synthetase.

Examples of angiostatic tRNA synthetase fragments having a methionine at their N-terminus include, but are not limited to those selected from the group consisting of SEQ ID NOS 15-17, 27-29, 36-38, 48-50 and any homologs and analogs thereof.

Examples of angiostatic tRNA synthetase fragments not having a methionine at their N-terminus include, but are not limited to those selected from the group consisting of SEQ ID NOS 12-14, 24-26, 36-38, 48-50, and any homologs and analogs thereof.

In any of the kits herein, a composition in the first contain may have a pI greater than 7.1.

Such kits may further include a second therapeutic agent, such as an antineoplastic agent, an anti-inflammatory agent, an antibacterial agent, an angiogenic agent, an antiviral agent, or an anti-angiogenic agent. The second therapeutic agent may be contained in a separate container.

In some embodiments, a kit of the present invention can include a container comprising an antibody that specifically binds to an epitope of a tRNA synthetase fragment and written instructions for use thereof. In such examples, the tRNA synthetase fragment can be a tryptophanyl tRNA synthetase fragment, a human tRNA synthetase fragment, and/or any angiostatic fragment of a tRNA synthetase. In some embodiments, an angiostatic tRNA synthetase framgment is one selected from the group consisting of SEQ ID NOS: 12-17, 24-29, 36-41, 48-53, and any homologs and analogs thereof.

The kits herein can also include one or more syringes or other delivery devices (e.g., stents, implantable depots, etc.). The kits can also include a set of written instructions for use thereof.

### EXAMPLES

### Example 1

Preparation of Endotoxin-Free Recombinant TrpRS

Endotoxin-free recombinant human TrpRS was prepared as follows. Plasmids encoding full-length TrpRS (amino acid residues 1-471 of SEQ ID NO: 1), or truncated TrpRS, hereinafter referred to as T2 (SEQ ID NO: 12), consisting essentially of residues 94-471 of SEQ ID NO: 1 (i.e., residues 94-471 of full-length TrpRS) and a second truncated TrpRS, hereinafter referred to as T1 (SEQ ID NO: 13), consisting essentially of residues 71-471 of SEQ ID NO: 1 were prepared. Each plasmid also encoded a C-terminal tag comprising six histidine residues (e.g. amino acid residues 472-484 of SEQ ID NO: 1), and an initial methionine residue. The His.sub.6-tagged T1 has the amino acid sequence of SEQ ID NO: 5, whereas the His.sub.6-tagged T2 has the amino acid sequence of SEQ ID NO: 7.

The above plasmids were introduced into E. coli strain BL 21 (DE 3) (Novagen, Madison, Wiss.). Human mature EMAPII, also encoding a C-terminal tag of six histidine residues, was similarly prepared for use. Overexpression of recombinant TrpRS was induced by treating the cells with isopropyl .beta.-D-thiogalactopyranoside for 4 hours. Cells were then lysed and the proteins from the supernatant purified on HIS.cndot.BIND.RTM. nickel affinity columns (Novagen) according to the manufacturer's suggested protocol. Following purification, TrpRS proteins were incubated with phosphate-buffered saline (PBS) containing 1 μM ZnSO.sub.4 and then free Zn²+ was removed (Kisselev et al., Eur. J. Biochem. 120:511-17 (1981)).

Endotoxin was removed from protein samples by phase separation using Triton X-114 (Liu et al., Clin. Biochem. 30:455-63 (1997)). Protein samples were determined to contain less than 0.01 units of endotoxin per mL using an E-TOXATE.RTM. gel-clot assay (Sigma, St. Louis, Mo.). Protein concentration was determined by the Bradford assay (Bio-Rad, Hercules, Calif.) using bovine serum albumin (BSA) as a standard.

### Example 2

Cleavage of Human TrpRS by PMN Elastase

Cleavage of human full-length TrpRS by PMN elastase was examined: TrpRS was treated with PMN elastase in PBS (pH 7.4) at a protease:protein ratio of 1:3000 for O, 15, 30, or 60 minutes. Following cleavage, samples were analyzed on 12.5% SDS-polyacrylamide gels. PMN elastase cleavage of a full-length TrpRS of about 53 kDa, encoded by nulceotides 3428 to 4738 of DNA SEQ ID NO: 2) generated a major fragment of about 46 kDa (SEQ ID NO: 5, T1 having the C-terminal histidine tag) and a minor fragment of about 43.5 kDa (SEQ ID NO: 7, T2 having the C-terminal histidine tag).

Western blot analysis with antibodies directed against the carboxyl-terminal His.sub.6-tag of the recombinant TrpRS protein revealed that both fragments possessed the His.sub.6-tag at their carboxyl-terminus. Thus, only the amino-terminus of two TrpRS fragments has been truncated. The amino-terminal sequences of the TrpRS fragments were determined by Edman degradation using an ABI Model 494 sequencer. Sequencing of these fragments showed that the amino-terminal sequences were S—N—H-G-P (SEQ ID NO: 8) and S-A-K-G-I (SEQ ID NO: 9), indicating that the amino-terminal residues of the major and minor TrpRS fragments were located at positions 71 and 94, respectively, of full-length TrpRS. These human TrpRS constructs are summarized in FIG. 1. Signature sequences -HVGH- (SEQ ID NO: 10) and -KMSAS- (SEQ ID NO: 11) are shown in boxes.

The angiostatic activity of the major and minor TrpRS fragments was analyzed in angiogenesis assays. Recombinant forms of the major and minor TrpRS fragments SEQ ID NO: 5 and SEQ ID NO: 7 each having a C-terminal histidine tag (amino acid residues 472-484 of SEQ ID NO: 1) were used in these assays. Both TrpRS fragments were capable of inhibiting angiogenesis.

### Example 3

Truncated Fragments of Trp-RS Show Potent Angiostatic Effect for Retinal Angiogenesis

Angiostatic activity of truncated forms derived from tryptophanyl-rRNA synthetase (TrpRS, 53 kDa; SEQ ID NO: 1) was examined, in a post-natal mouse retinal angiogenesis model Friedlander et al. Abstracts 709-B84 and 714-B89, IOVS 41(4): 138-139 (Mar. 15, 2000) has reported that postnatal retinal angiogenesis proceeds in stages in the mouse. The present invention provides a method of assaying angiogenesis inhibition by exploiting this staged retinal vascularization.

Endotoxin-free recombinant mini-TrpRS (48 kDa splice variant of histidine tagged TrpRS; SEQ ID NO: 3) and T2 (approximately 43 kDa cleavage product of histidine tagged TrpRS; SEQ ID NO: 7) were prepared as recombinant proteins. These proteins were injected intra-vitreally into neonatal Balb/C mice on postnatal (P) day 7 or 8 and the retinas harvested on P12 or P13. Collagen IV antibody and fluorescein-conjugated secondary antibody were used to visualize the vessels in retinal whole mount preparations. Anti-angiogenic activity was evaluated by confocal microscopic examination based upon the effect of injected proteins on formation of the deep, outer, vascular plexus. Intra-vitreous injection and retina isolation was performed with a dissecting microscope (SMZ 645, Nikon, Japan). An eyelid fissure was created in postnatal day 7 (P7) mice with a fine blade to expose the globe for injection of T2 (5 pmol) or TrpRS (5 pmol). The samples (0.5 μl) were injected with a syringe fitted with a 32-gauge needle (Hamilton Company, Reno, Nev.). The injection was made between the equator and the corneai limbus; during injection the location of the needle tip was monitored by direct visualization to determine that it was in the vireous cavity. Eyes with needle-induced lens or retinal damage were excluded from the study. After the injection, the eyelids were repositioned to close the fissure.

On postnatal day 12 (P12), animals were euthanized and eyes enucleated. After 10 minutes in 4% paraformaldehyde (PFA) the cornea, lens, sclera, and vitreous were excised through a limbal incision. The isolated retina was prepared for staining by soaking in methanol for 10 minutes on ice, followed by blocking in 50% fetal bovine serum (Gibco, Grand Island, N.Y.) with 20% normal goat serum (The Jackson Laboratory, Bar Harbor, Me.) in PBS for 1 hour on ice. The blood vessels were specifically visualized by staining the retina with a rabbit anti-mouse collagen IV antibody (Chemicon, Temecula, Calif.) diluted 1:200 in blocking buffer for 18 h at 4° C. An ALEXA FLUOR.RTM. 594-conjugated goat anti-rabbit IgG antibody (Molecular Probes, Eugene, Oreg.) (1:200 dilution in blocking buffer) was incubated with the retina for 2 h at 4° C. The retinas were mounted with slow-fade mounting media M (Molecular Probes, Eugene, Oreg.).

Angiostatic activity was evaluated based upon the degree of angiogenesis in the deep, outer retinal vascular layer (secondary layer) that forms between P8 and P12. The appearance of the inner blood vessel network (primary layer) was evaluated for normal development and signs of toxicity. None of the protein constructs used in this example produced any adverse effects on the primary layer.

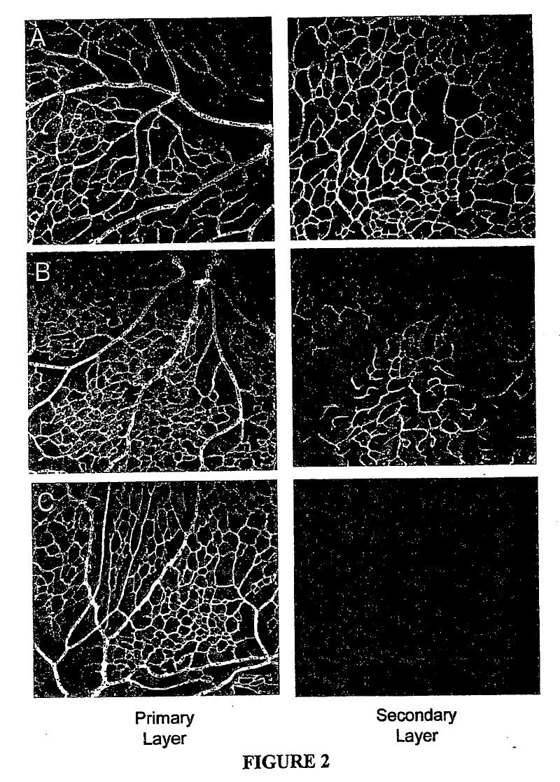

FIG. 2 provides a photomicrographic depiction of the ability of T2 to inhibit vascularization of the secondary deep network of the mouse retina. In FIG. 2, row A shows the vascular network of a retina exposed to TrpRS, Row B shows the vascular network of a retina exposed to Mini-TrpRS, and row C shows the vascular network of a retina exposed to polypeptide T2 of the present invention. The first (left) column shows the primary superficial network, and the second column shows the secondary deep network. As is evident from FIG. 2, none of the polypeptides affected the primary superficial network, whereas only T2 significantly inhibited vascularization of the secondary deep network.

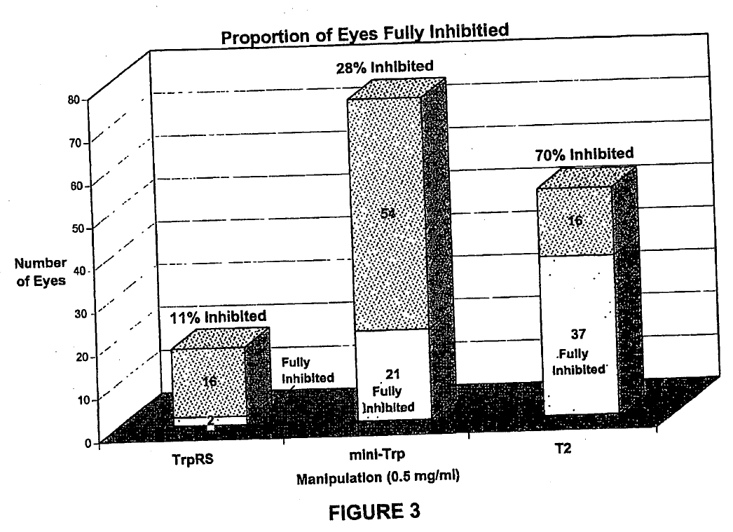

Most PBS-treated eyes exhibited normal retinal vascular development, but complete inhibition of the outer vascular layer was observed in about 8.2% (n=73) of the treated eyes. Complete inhibition of the outer network was observed in 28% of mini-TrpRS (0.5 mg/mL)-treated eyes (n=75). The smaller, truncated form (T2) was a far more potent inhibitor of angiogenesis in a dose dependent fashion; 14.3% were completely inhibited after treatment with 0.1 mg/mL of T2 (n=14), 40% after treatment with 0.25 mg/mL (n=20) and 69.8% inhibited completely after 0.5 mg/mL (n=53). The data for the 0.5 mg/mL treatments are presented graphically in FIG. 3. Extracts of mouse retina contain a protein with the same apparent molecular mass and immunoreactivity as human mini-TrpRS, as analyzed by SDS-PAGE and Western Blot. Full-length mouse and human TrpRS share about 88% amino acid identity and contain 475 and 471 amino acids, respectively. Truncated forms of TrpRS, especially T2, have a potent angiostatic effect on retinal vascular development.

### Example 4

Matrigel Angiogenesis Assay.

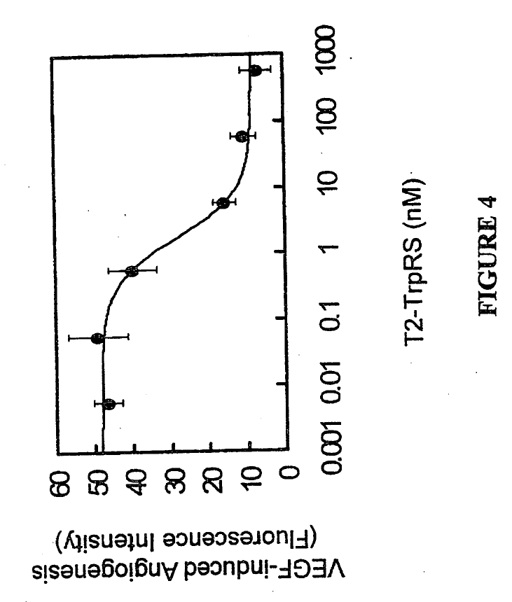

A mouse matrigel angiogenesis assay was used to examine the angiostatic activity of T2 (SEQ ID NO: 7) according to the methods described by Brooks et al. Methods Mol. Biol., 129: 257-269 (1999) and Eliceiri et al. Mol. Cell, 4: 915-924 (1999). It was performed as described with the following modifications. Athymic wehi mice were subcutaneously implanted with 400 μl growth-factor depleted matrigel (Becton Dickinson, Franklin Lakes, N.J.) containing 20 nM VEGF. The angiostatic activity of T2 was initially tested by including 2.5 μM T2 in the matrigel plug. The potency was determined by including various concentrations of T2 in the plug. On day 5, the mice were intravenously injected with the fluorescein-labeled endothelial binding lectin Griffonia (Bandeiraea) Simplicifolia I, isolectin B4 (Vector Laboratories, Burlingame, Calif.) and the matrigel plugs were resected. The fluorescein content of each plug was quantified by spectrophotometric analysis after grinding the plug in RIPA buffer (10 mM sodium phosphate, pH 7.4, 150 mM sodium chloride, 1% Nonidet P-40, 0.5% sodium deoxycholate, 0.1% sodium dodecyl sulfate). The data in Example four is illustrated in FIG. 4.

### Example 5

Localization of T2 Binding within the Retina.

To assess the uptake and localization of T2 injected into the retina, fluorescein-labeled (ALEXA.RTM. 488, Molecular Probes, Inc., Eugene Oreg.) T2 was injected into the vitreous of the eye on postnatal day 7 (P7). Globes were harvested on P8 and P 12 and fixed in 4% PFA for 15 min. The retinas were further dissected free of adherent nonretinal tissue and placed in 4% PFA overnight at 4° C. and then embedded in medium (TISSUE-TEK.RTM. O.C.T., Sakura FineTechnical Co., Japan) on dry ice. Cryostat sections (10 micron) were rehydrated with PBS and blocked with 5% BSA, 2% normal goat serum in PBS. Blood vessels were visualized with anti-mouse collagen IV antibody as described above. VECTASHIELD.RTM. containing DAPI nuclear stain (Vector Laboratories, Burlingame, Calif.) was used to mount the tissues with a cover slip.

Alternatively, unstained retina sections were incubated with 200 nM fluorescein-labeled full-length TrpRS or fluorescein-labeled T2 in blocking buffer overnight at 4° C. Sections were washed six times for 5 minutes each in PBS, followed by incubation with 1 μg/mL DAPI for 5 minutes for visualization of the nuclei. Pre-blocking with unlabeled T2 was performed by incubating 1 μM unlabeled T2 for 8 hours at 4° C. prior to incubation with fluorescein-labeled T2. Retinas were examined with a multiphoton BioRad MRC1024 confocal microscope. 3-D vascular images were produced from a set of Z-series images using the Confocal Assistant software (BioRad, Hercules, Calif.).

Angiostatic Potency of T2 in the Mouse Matrigel Plug Assay. We examined T2 (SEQ ID NO: 7) to determine whether it had angiostatic activity, even though it had lost aminoacylation activity. The mouse matrigel assay was used to examine the angiostatic activity of T2 in vivo. VEGF₁₆₅-induces the development of blood vessels into the mouse matrigel plug. When T2 was added to the matrigel along with VEGF₁₆₅, angiogenesis was blocked in a dose-dependent manner with a IC₅₀ of 1.7 nM as shown in FIG. 4.

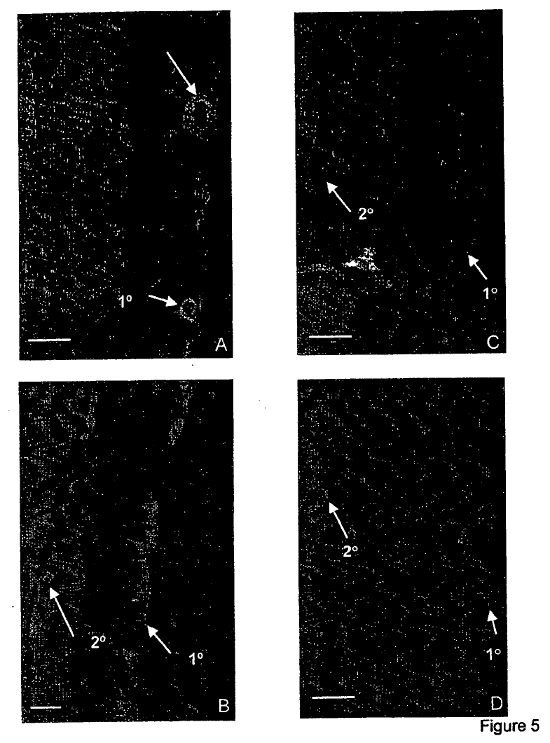

Fluorescein-labeled T2 Localizes to Retinal Blood Vessels. In order to visualize the intraocular localization of T2 (SEQ ID NO: 7), we examined the distribution of fluorescein-labeled T2 following intravitreous injection on postnatal day 7. Retinas were isolated the following day, sectioned and examined using confocal microscopy. The distribution of the injected protein was restricted to blood vessels. This localization was confirmed by co-staining labeled T2 treated eyes with an fluorescein-labeled (ALEXA.RTM. 594) anti-collagen IV antibody (data not shown). Five days after injection of fluorescein-labeled T2 (on P12), the green fluorescence of the labeled T2 was still visible (FIG. 5A). In these retinas, no secondary vascular layer was observed at P12, indicating that the fluorescein-labeled T2 retained angiostatic activity comparable to unlabeled T2. Retinas injected on P7 with fluorescein-labeled full-length TrpRS developed a secondary vascular layer by P12 but no vascular staining was observed (FIG. 5B). In FIG. 5, fluorescein-labeled proteins are green, collagen-labeled vessels are red, and nuclei are blue.

To further evaluate the binding properties of labeled T2, cross-sectioned slices. of normal neonatal retinas were stained with fluorescein-labeled T2. Under these conditions, fluorescein-labeled T2 only bound to blood vessels (FIG. 5C). The binding was specific as it was blocked by pre-incubation with unlabeled T2 (data not shown). No retinal vessel staining was observed when fluorescein-labeled full-length TrpRS was applied to the retinas (FIG. 5D), consistent with the absence of angiostatic activity of the full-length enzyme.

As shown in FIG. 5, fluorescein-labeled T2 is angiostatic and localizes to retinal blood vessels. Fluorescein-labeled T2 (FIG. 5A) or full-length TrpRS (FIG. 5B) were injected (0.5 μl, intravitreous) on postnatal day 7 (P7). The retinas were harvested on P8 and stained with an anti-collagen IV antibody and DAPI nuclear stain, Labeled T2 (upper arrow pointing to vessel in FIG. 5A) localized to blood vessels in the primary superficial network (1°). Note that the secondary deep network is completely absent (2°). While both the primary (1°) and secondary (2°) vascular layers are present in eyes injected with fluorescein-labeled full-length TrpRS (arrows in FIG. 5B), no labeling is observed.

In a separate set of experiments, frozen sections of P15 retinas were stained with fluorescein-labeled T2 (FIG. 5C) or fluorescein-labeled full-length TrpRS (FIG. 5D) and imaged in the confocal scanning laser microscope. Labeled T2 selectively localized to blood vessels and appears as a bright green vessel penetrating the primary and secondary retinal vascular layers just below the label “2°” in FIG. 5C. No staining was observed with full-length TrpRS (FIG. 5D).

Full-length TrpRS contains a unique NH₂-terminal domain and lacks angiostatic activity. Removing part or all of this entire domain reveals a protein with angiostatic activity. The NH₂-terminal domain, which can be deleted by alternative splicing or by proteolysis, may regulate the angiostatic activity of TrpRS, possibly by revealing a binding site necessary for angiostasis that is inaccessible in full-length TrpRS.

VEGF-induced angiogenesis in the mouse matrigel model was completely inhibited by T2 as was physiological angiogenesis in the neonatal retina. Interestingly, the most potent anti-angiogenic effect of TrpRS fragments in vitro and in CAM and matrigel models is observed in VEGF-stimulated angiogenesis. The neonatal mouse retinal angiogenesis results are consistent with a link between VEGF-stimulated angiogenesis and the angiostatic effects of TrpRS fragments; retinal angiogenesis in this system may be driven by VEGF. In addition, the inhibition observed in the retinal model was specific for newly developing vessels; pre-existing (at the time of injection) primary vascular layer vessels were unaltered by the treatment. While the mechanism for the angiostatic activity of T2 is not known, the specific localization of T2 to the retinal endothelial vasculature and the selective effect of T2 on newly developing blood vessels suggest that T2 may function through an endothelial cell receptor expressed on proliferating or migrating cells. Further understanding of the mechanism of T2 angiostatic activity requires more detailed identification of the mechanism of action.

A variety of cell types that produce, upon interferon-γ stimulation, the angiostatic mini TrpRS also produce angiostatic factors such as IP-10 and MIG. Thus, these results raise the possibility of a role for TrpRS in normal, physiologically relevant pathways of angiogenesis. Another ubiquitous cellular protein—pro-EMAPI (p43)—has two apparently unrelated roles similar to those reported here for TrpRS. Pro-EMAPII assists protein translation by associating with the multisynthetase complex of mammalian aminoacyl tRNA synthetases. It is processed and secreted as EMAPII, and a role for EMAPII as an angiostatic mediator during lung development has been suggested.

Thus, T2 can be utilized in physiologically relevant angiogenic remodeling observed under normal or pathological conditions. In normal angiogenesis, T2 can aid in establishing physiologically important avascular zones present in some organs such as the foveal avascular zone of the central retina. Pathological angiogenesis can occur if the cleavage of full-length TrpRS was inhibited, leading to an overgrowth of vessels.

In ocular diseases, neovascularization can lead to catastrophic loss of vision. These patients can potentially receive great benefit from therapeutic inhibition of angiogenesis. Vascular endothelial growth factor has been associated with neovascularization and macular edema in the retina although it is believed that other angiogenic stimuli also have roles in retinal angiogenesis. We have observed an association between VEGF-stimulated angiogenesis and potent angiostatic activity of TrpRS fragments, making these molecules useful in the treatment of hypoxic, and other, proliferative retinopathies. There has been no report in the literature of an anti-angiogenic agent that completely inhibits angiogenesis 70% of the time, as does the T2 of the present invention (FIG. 5). Another advantage of TrpRS fragments is that they represent naturally occurring and, therefore, potentially non-immunogenic, anti-angiogenics. Thus, these molecules can be delivered via targeted cell- or viral vector-based therapy. Because many patients with neovascular eye diseases have associated systemic ischemic disease, local anti-angiogenic treatment with genetically engineered cells or viral vectors placed directly into the eye is desirable.

In addition to treatment of angiogenic retinopathies, the TrpRS fragments of the present invention, particularly T2 and angiogenesis inhibiting fragments thereof, can also inhibit solid tumor growth by preventing vacularization of the tumor. The TrpRS fragments of the present invention block VEGF-induced proliferation and chemotaxis of endothelial cells in vitro, and are thus useful in the treatment of any pathology involving unwanted endothelial cell proliferation and vascularization.

### Example 6

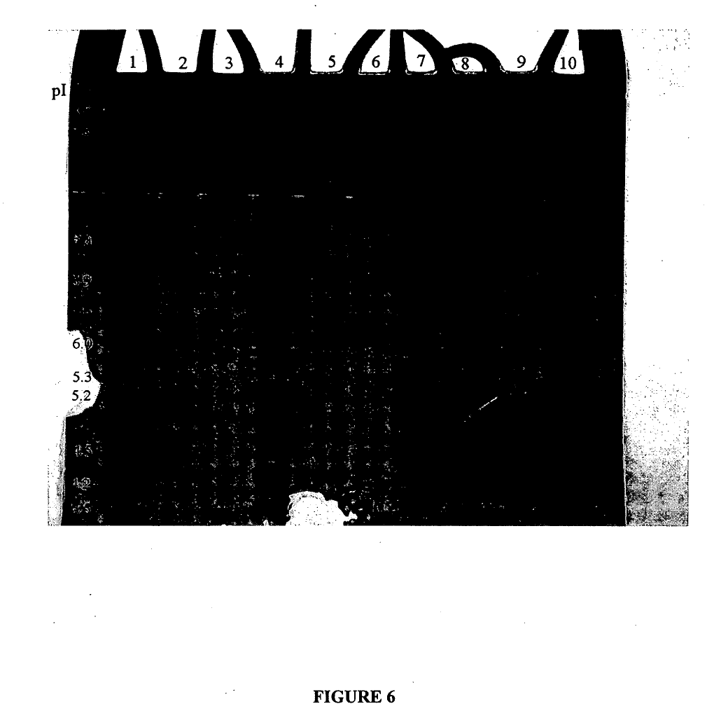

FIG. 6 illustrates the measurement of pI (the effective charge) of a compound (e.g., a multi-unit complex) produced by a prokaryotic cell transfected with an amino acid sequence of SEQ ID NO: 15. The following Table 1 is a summary of each lane.

TABLE 1

| Lane No. | Sample | Load | |
|---|---|---|---|
| 1 | Marker | 5 | μL |
| 2 | Reference 1 | 1 | μg |
| 3 | Reference 2 | 1 | μg |
| 4 | Marker | 5 | μL |
| 5 | Reference 2 | 2 | μg |
| 6 | Reference 1 | 2 | μg |
| 7 | Marker | 5 | μL |
| 8 | Reference 2 | 4 | μg |
| 9 | Reference 1 | 4 | μg |
| 10 | Marker | 5 | μL |

Reference 1 and Reference 2 were both prepared using the same clone, but at different sites. Samples were diluted 1:1 with Novex pH 3-10 sample buffer. The marker used with an IEF Marker from Invitrogen™.

While the theoretical pI for T2 (e.g., SEQ ID NO: 12 or 15) should be 7.1, the effective pI for a polypeptide produced recombinantly using an expression vector encoding a polypeptide having SEQ ID NO: 15, was measured at about 7.6, as is illustrated by FIG. 6. This suggests that some of the negative charges of the primary sequence are “hidden” or inaccessible to the local environment.

### Example 7

Cell Disruption and Clarification of Lysate

In the following cell disruption and lysate clarification procedure, all steps were performed at 4° C. and the pH of all buffers adjusted at 4° C.

The total mass of cell paste collected from the fermentation tank was divided into seven batches, each batch containing approximately 90 g of cell paste. The cell paste for each batch was mixed for approximately 3 minutes with 950 mL of cold Lysis Buffer (25 mM Tris pH 8.0, 10% Glycerol, 1 mM EDTA) using a homogenizer.

The suspension for each batch was then passed twice through an Avestin EmulsiFlex C-50 high pressure homogenizer at 10,000 to 20,000 PSI and collected on ice, taking care that the temperature of the lysate did not exceed 10° C. The homogenizer was then flushed with lysis buffer to remove the residual lysate.

The lysate (˜1150 mL) for each batch was then centrifuged at 38,250 g for 55 minutes. The supernatant (˜1100 mL) was retained and the pellets were discarded. For each batch, the supernatant was loaded on the Q sepharose HP column as quickly as possible (see Q Sepharose chromatography). All of the above steps for the cell disruption and clarification for any additional batches of cells was performed, followed by immediate loading onto the Q Sepharose column following clarification.

Q Sepharose Chromatography

The supernatant from the centrifugation process was loaded onto a 2.2 L (13 cm diameter, 16.6 cm height) Q Sepharose High Performance column. The column load of the protein should not exceed 5 mL of lysate per mL of resin. The column was pre-equilibrated with 2.5 L of Buffer B (25 mM Tris pH 8.0, 10% glycerol, 1M NaCl) followed by 11 L of Buffer A (25 mM Tris pH 8.0, 10% glycerol). The load flow rate (for the soluble material) was 20-50 mL/min (˜10-25 cm/hr) and the column flow through collected.

The column was washed with 30 column volumes (66 L) of Buffer A at 60 mL/min (˜30 cm/hr). The column was then eluted with a 20 column volume (44 L) linear gradient, from Buffer A to 20% Buffer B at 100 mL/min (˜50 cm/hr) and 500 mL fractions were collected during the elution peak (‘Q fractions’). The Q fractions were analyzed by SDS-PAGE (for both the amount of T2-TrpRS in the fraction and the relative purity of the material) and the fractions containing the greatest amounts of purified T2-TrpRS were pooled. Reverse-phase HPLC represents one possible alternative to the use of SDS-PAGE for fraction analysis.

Endotoxin Reduction Filtration

The total pool of Q fractions was filtered at 4° C. through 2 Pall endotoxin reduction filtration cartridges with a Mustang E membrane at 10 mL/min, collecting the flow through. The sample was split between the two filter cartridges and only exposed to the filter membrane once. Approximately 93% of the total protein was recovered following the endotoxin reduction filtration.

Concentration and Buffer Exchange

The endotoxin reduction filtered pool (8500 mL) was concentrated to <1 L using a Cross-Flow (Ultrafiltration) filter (molecular weight cut off of 10,000) at pressures of 5-7 psi. The filtrate was collected and checked by Bradford assay for leaking polypeptide. The concentrated pool (<1 L) was diluted five-fold with CM Buffer A (25 mM HEPES pH 8.0, 10% glycerol) to increase the volume of the sample to 5L. The conductivity of the final dilution pool was 1.02 mS, whereas the conductivity of the CM Buffer A was 0.74 mS.

CM Sepharose Chromatography

The sample from the buffer exchange process was loaded onto a 1300 mL (13 cm diameter, 9.8 cm height) CM Sepharose Fast Flow column, pre-equilibrated with 6.5 L of Buffer A (25 mM HEPES pH 8.0, 10% glycerol); the load flow rate was 90 mL/min (˜40 cm/hr). The column was washed with 15 column volumes (19.5 L) of Buffer A at 70 mL/min (˜30 cm/hr). The column was then eluted with a 20 column volume (26 L) linear gradient, from Buffer A to 50% Buffer B (25 mM HEPES pH 8.0, 10% glycerol, 1M NaCl) at 100 mL/min (˜50 cm/hr) and 500 mL fractions were collected during the elution peak. The CM fractions were analyzed by SDS-PAGE (for both the amount of T2-TrpRS in the fraction and the relative purity of the material), and fractions containing the greatest amounts of purified T2-TrpRS were pooled. Reverse-phase HPLC represents one possible alternative to the use of SDS-PAGE for fraction analysis.

Final Sample Concentration and Buffer Exchange

The pooled CM fractions (5500 mL) were concentrated to ˜150 mL using a Cross-Flow (Ultrafiltration) filter (molecular weight cut off of 10,000 dalton) at pressures of 2-7 psi. The filtrate fractions were collected and checked by Bradford assay for leaking polypeptide. The concentrated pool (145 mL) was dialyzed against 15 L of final storage buffer (5 mM sodium phosphate pH 7.4, 150 mM NaCl, 50% glycerol) using dialysis tubing having a 6-8000 dalton molecular weight cut off at 4° C. (˜16 hours).

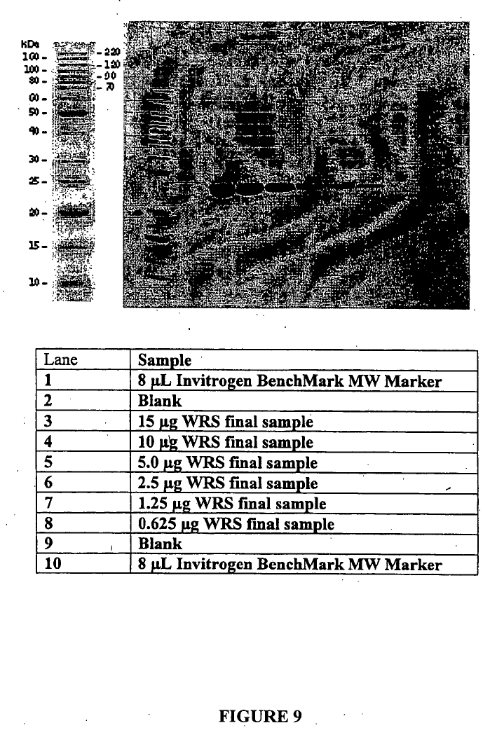

The dialyzed pool (˜50 mL) was removed from dialysis and assays were completed on the sample. The final volume of the concentrated sample was 52 mL and the final concentration of the sample was 26.3 mg/mL based on the standard Bradford assay. Final denaturing SDS-PAGE analysis of the sample was completed for a purity determination. See FIG. 9.

TABLE 2
ANALYSIS OF T2 SAMPLES

| | | | Total | | |
|---|---|---|---|---|---|
| | Volume | Protein | Protein | Recovery | Purity |
| Fraction | (mL) | (mg/mL) | (mg) | % | % |
| Q HP pool | 8500 | 0.4 | 3400 | — | >85% |
| Q HP pool post | 8800 | 0.36 | 3168 | 93% | >85% |
| endotoxin filter | | | | | |
| CM load | 4800 | 0.663 | 3182 | 93.6% | >85% |
| CM pool | 5500 | 0.345 | 1897.5 | 55.8% | >95% |
| CM pool | 145 | 13.36 | 1937.2 | 57% | >95% |
| concentrated | | | | | |
| Final sample | 52 | 26.3 | 1367.6 | 40.2% | >95% |

The endotoxin level of this sample, measured using a PyroGene™ endotoxin assay from Invitrogen Corporation, was determined to be 6.25 EU/mg of protein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising an isolated tRNA synthetase fragment consisting essentially of an amino acid sequence selected from the group consisting of SEQ ID NOS: 15-17 and any homologs and analogs thereof.

2. The composition of claim 1 wherein said amino acid sequence is SEQ ID NO: 15.

3. The composition of claim 2 wherein said tRNA synthetase fragment is less than 45 kD.

4. The composition of claim 2 wherein said tRNA synthetase fragment is anti-angiogenic.

5. The composition of claim 2 wherein said tRNA synthetase fragment is purified.

6. The composition of claim 1 wherein said amino acid sequence is SEQ ID NO: 16.

7. The composition of claim 6 wherein said tRNA synthetase fragment is less than 48 kD.

8. The composition of claim 6 wherein said tRNA synthetase fragment is anti-angiogenic.

9. The composition of claim 6 wherein said tRNA synthetase fragment is purified.

10. The composition of claim 1 wherein said amino acid sequence is SEQ ID NO: 17.

11. The composition of claim 10 wherein said tRNA synthetase fragment is less than 53 kD.

12. The composition of claim 10 wherein said tRNA synthetase fragment is anti-angiogenic.

13. The composition of claim 10 wherein said tRNA synthetase fragment is purified.

14. A pharmaceutical formulation comprising the composition of claim 1 and a pharmaceutically acceptable carrier.

15. A composition comprising an isolated tRNA synthetase fragment comprising of an amino acid sequence selected from the group consisting of SEQ ID NOS: 24-29, 36-41, 48-53, and any homologs and analogs thereof.

16. The composition of claim 15 wherein said amino acid sequence is selected from the group consisting of SEQ ID NOS: 24, 27, 36, 39, 48, and 51.

17. The composition of claim 16 wherein said tRNA synthetase fragment is less than 45 kD.

18. The composition of claim 16 wherein said tRNA synthetase fragment is anti-angiogenic.

19. The composition of claim 16 wherein said tRNA synthetase fragment is purified.

20. The composition of claim 15 wherein said amino acid sequence is selected from the group consisting of SEQ ID NOS: 25, 28, 37, 40, 49, and 52.

21. The composition of claim 20 wherein said tRNA synthetase fragment is less than 48 kD.

22. The composition of claim 20 wherein said tRNA synthetase fragment is anti-angiogenic.

23. The composition of claim 20 wherein said tRNA synthetase fragment is purified.

24. The composition of claim 15 wherein said amino acid sequence is selected from the group consisting of SEQ ID NOS: 26, 29, 38, 41, 50, and 53.

25. The composition of claim 24 wherein said tRNA synthetase fragment is less than 53 kD.

26. The composition of claim 24 wherein said tRNA synthetase fragment is anti-angiogenic.

27. The composition of claim 24 wherein said tRNA synthetase fragment is purified.

28. A pharmaceutical formulation comprising the composition of claim 15 and a pharmaceutically acceptable carrier.

29. The composition of claim 1 or 15 wherein said tRNA synthetase fragment is purified such that concentration of endotoxins in said composition is less than 10 endotoxin units per milligram of said tRNA synthetase fragment.

30. The composition of claim 1 or 15 having a pI greater than 7.1.

31. A method for modulating angiogenesis in a cell or an organism comprising administering to said cell or said organism a composition of claim 1 or 15.

32. A method for treating a patient suffering from an angiogenic condition comprising administering to said patient a composition of claim 1 or 15.

33. A kit comprising a container wherein said container contains a composition of claim 1 or 15 and written instruction for use thereof in modulating angiogensis.
